(19) United States
(12) Patent Application Publication
(10) Pub. No.: US 2022/0177542 A1
(43) Pub. Date: Jun. 9, 2022

(54) MHC CLASS II MOLECULE-BASED PEPTIDE EXCHANGE SYSTEM AND METHOD

(71) Applicant: MBL International Corp., Woburn, MA (US)

(72) Inventors: Marc Delcommenne, Chicago, IL (US); Yuri Poluektov, Woburn, MA (US)

(21) Appl. No.: 17/112,570

(22) Filed: Dec. 4, 2020

(51) Int. Cl. C07K 14/74; C07K 16/24; C07K 14/36; G01N 33/563; G01N 33/553; G01N 33/543; G01N 33/533; G01N 33/535; G01N 33/534

(52) U.S. Cl. CPC C07K 14/70539; C07K 16/247; C07K 14/36; G01N 33/563; C07K 2319/20; G01N 33/54326; G01N 33/533; G01N 33/535; G01N 33/534; G01N 33/553

(57) ABSTRACT

The present disclosure relates to compositions, kits, and methods to perform peptide exchange on MHC class II molecules, such as quantified peptide exchange.

### CROSS-REFERENCE TO RELATED APPLICATIONS

This application claims the benefit of U.S. Provisional Application No. 62/944,998, filed Dec. 6, 2019, which is incorporated by reference herein in its entirety.

### BACKGROUND OF THE INVENTION

CD4+ T cells play an important role in initiating both humoral and cellular immune responses. Deficiencies in CD4+ T cell function can be life threatening as seen in AIDS and cancer patients. As CD4+ T cells are functionally highly heterogeneous because of their antigen specificity, their functional characterization requires multiple reliable reagents. One approach for analyzing CD4+T lymphocytes specific to a particular allele/antigen combination consists in MHC class II tetramer staining and multicolor flow cytometry analysis. MHC class II tetramers are the assembly of four MHC class II extracellular alpha and beta chains whose pairing is made possible by the addition of a leucine zipper peptide at their COOH-terminus. Alpha/beta complexes, or raw monomers, loaded with peptides of interest are biotinylated at the C-terminus of the alpha chain before being tetramerized with fluorochrome conjugated streptavidin. Streptavidin can alternatively be conjugated with metals, DNA barcodes or other molecules.

Development of vaccines or T cell based immunotherapies often requires to test a large number of neoantigen peptides for MHC class II binding and T cell characterization. Systems that can perform both tasks are needed.

### SUMMARY OF THE INVENTION

The present disclosure relates to compositions, kits, and methods to perform peptide exchange on MHC class II molecules, such as quantified peptide exchange. They may be exchanged in any multimeric state, such as monomers, tetramers, octamers, or dodecamers without the use of any catalyst or peptide exchange factor of any kind. The methods of the present disclosure allow MHC II proteins with exchanged peptides to be used in further applications such as cell staining. The methods may also be used to quantify peptides present in complex mixtures.

In one aspect, the present disclosure provides methods to perform peptide exchange on MHC class II molecules.

In another aspect, the present disclosure provides methods for assessing binding affinity of peptides towards MHC class II in a semi-quantitative manner.

In another aspect, the present disclosure provides MHC class II tetramers/monomers with high peptide exchangeability, and methods for producing them.

In another aspect, the present disclosure provides MHC class II tetramers/monomers with exchanged peptides.

In another aspect, the present disclosure provides compositions and kits for performing the methods disclosed herein.

In one aspect, the present disclosure provides a protein complex comprising an MHC class II molecule bound to a first peptide, wherein the first peptide is labeled with a first label. In one embodiment, the protein complex can be used for quantified peptide exchange. In another embodiment, the protein complex can be used for non-quantified peptide exchange.

In another aspect, the present disclosure provides a kit for peptide exchange comprising an MHC class II molecule bound to a first peptide wherein the first peptide is labeled with a first label. In one embodiment, the kit is used for quantified peptide exchange. In another embodiment, the kit is used for non-quantified peptide exchange. In still another embodiment, the kit does not comprise any catalyst or peptide exchange factor. In yet another embodiment, the kit further comprises an anti-first tag antibody. In another embodiment, the kit further comprises a capture system comprising an anti-MHC class II protein antibody or a streptavidin. In still another embodiment, the capture system comprises magnetic capture beads. In yet another embodiment, the kit further comprises a reference peptide.

In another aspect, the present disclosure provides a method of performing peptide exchange on an MHC class II molecule, comprising: providing an MHC class II molecule bound to a first peptide, wherein the first peptide is labeled with a first label; contacting the MHC class II molecule bound to the first peptide with a second binding partner, wherein the contacting step generates an MHC class II molecule bound to the second binding partner.

In another embodiment, the method described herein further comprises providing a first quantity of MHC class II molecules bound to a first peptide, wherein the first peptide is labeled with a first label; adding to the MHC class II molecules bound to the first peptide a second quantity of a second binding partner, whereby a mixture is formed comprising MHC class II molecules bound to the first peptide, MHC class II molecules bound to the second binding partner, unbound first peptide, and unbound second binding partner.

In one embodiment, the method is for quantified peptide exchange. In another embodiment, the method is for non-quantified peptide exchange. In one embodiment, the method does not comprise adding any catalyst or peptide exchange factor.

In one embodiment, the second binding partner is a second peptide. In another embodiment, the second binding partner or second peptide is not labeled. In still another embodiment, the second binding partner or second peptide does not display a tag. In yet another embodiment, the second binding partner or second peptide displays a second label, and further wherein the first label is different from the second label. In another embodiment, the second label is selected from biotin, a hapten, or a tag. In still another embodiment, the second label is a second tag, and the first tag is different from the second tag.

In one embodiment, the method described herein further comprises determining: (1) at least one of: the amount of MHC class II molecules bound to the first peptide, the amount of MHC class II molecules bound to the second binding partner or second peptide, the amount of unbound first peptide, or the amount of unbound second binding partner or second peptide; (2) the ratio of the amount of the MHC class II molecules bound to the first peptide to the amount of the MHC class II molecules bound to the second binding partner or second peptide; and/or (3) the amount of unbound first peptide and comparing the amount of unbound first peptide to the first quantity of MHC class II molecules bound to the first peptide.

In one embodiment, the determining step comprises or is accomplished by performing a sandwich immunoassay comprising: providing a support conjugated to a first antibody or a molecule that binds to an MHC class II molecule such as streptavidin; binding the first antibody or a molecule that binds to an MHC class II molecule to the MHC class II molecules bound to the first peptide and the MHC class II molecules bound to the second binding partner or second peptide; providing a second antibody capable of binding the first peptide; binding the second antibody to the MHC class II molecules bound to the first peptide, whereby the second antibody is bound to the support; and determining the amount of the second antibody that is bound to the support.

In one embodiment, the support comprises magnetic beads, nonmagnetic beads, or microplate wells. In another embodiment, performing the sandwich immunoassay comprises pelleting beads using a magnet, by centrifugation, or by suction. In still another embodiment, the first antibody is an anti-MHC class II or anti-streptavidin antibody. In yet another embodiment, the first peptide is labeled with DNP and the second antibody is an anti-DNP antibody. In another embodiment, the second antibody is labeled with a fluorophore, an enzyme, a metal, barcode, or a radiolabel. In still another embodiment, the second antibody is labeled with a fluorophore. In yet one embodiment, the method further comprises conducting a control peptide exchange. In another embodiment, the mixture further comprises additional peptides, lipids, or polynucleotides.

Numerous embodiments are further provided that can be applied to any aspect of the present invention (e.g., any of the compositions, kits, and methods described above) and/or combined with any other embodiment described herein. For example, in one embodiment, the first peptide is PKPVSKMRMATPLLM, PKPVSLMRMATPLLM, KPVSKMRMARPLLMQ, PKPVSKYRMATPLLM, or a functional homologue thereof having at least 75% (e.g., at least 75%, 80%, 85%, 90%, 95%, 96%, 97%, 98%, or 99%) identity with PKPVSKMRMATPLLM, PKPVSLMRMATPLLM, KPVSKMRMARPLLMQ, or PKPVSKYRMATPLLM. In another embodiment, the first peptide is PKPVSKMRMATPLLM, PKPVSLMRMATPLLM, KPVSKMRMARPLLMQ, or PKPVSKYRMATPLLM.

In another embodiment, the first label is biotin, a hapten, or a tag. In another embodiment, the first label is a first tag. In another embodiment, the first tag is DNP. For example, and without limitation, such tagged peptides may include PK(DNP)PVSKMRMATPLLM, PK(DNP)PVSLMRMPTPLLM, K(DNP)PVSKMRMARPLLMQ, or PK(DNP)PVSKYRMATPLLM.

In another embodiment, the MHC class II molecule is a monomer. In another embodiment, the MHC class II molecule is a component of an MHC class II multimer. The MHC class II multimer may comprise 4, 6, or 12 MHC class II molecules. In another embodiment, at least one MHC class II molecule of the MHC class II multimer is bound to the first peptide. In another embodiment, all MHC class II molecules of the MHC class II multimer are bound to the first peptide.

In another embodiment, the MHC class II molecule is HLA-DPA1, HLA-DPB1, HLA-DQA1, HLA-DQB1, HLA-DRA, and HLA-DRB1, or I-A and I-E in mice. In another embodiment, the MHC class II molecule is HLA-DRB1*01:01, HLA-DRB1*04:01 or HLA-DRB1*15:01.

### BRIEF DESCRIPTION OF THE DRAWINGS

FIG. 1 shows a schematic diagram of peptide exchange and quantitation of peptide exchange on MHC class II monomers.

FIG. 2A-FIG. 2D and FIG. 3 show flow cytometry analysis of peptide displacement in a class II biotinylated monomer.

FIG. 4 shows a schematic diagram of peptide exchange and quantitation of peptide exchange on MHC class II tetramers.

FIG. 5A-FIG. 5E and FIG. 6 show flow cytometry analysis of peptide displacement in a class II tetramer.

FIG. 7 shows CD4+ T cell staining with peptide exchanged DR1 tetramers. Donor 213 PBMCs, 50 M cells total, Class II peptide stimulation. Day 15 10% human AB serum block, Staining with Desatinib+Fresh QS Tetramer Overnight (10 mM O/N), prepared 1 mM QS Tetramer (1 mM) or prepared 100 μM QS Tetramer (100 μM) 2 hr, 37° C. followed by 20 min RT (BioGems CD3 & CD4).

FIG. 8 shows comparison of peptide exchange rate in HLA-DRB1*01:01 tetramer and theoretical peptide binding affinities to DRB1.

FIG. 9 shows comparison of peptide exchange rate in HLA-DRB1*04:01 monomer and theoretical peptide binding affinities to DRB4.

FIG. 10 shows comparison of peptide exchange rate in HLA-DRB1*15:01 monomer and theoretical peptide binding affinities to DRB15.

FIG. 11 shows comparison peptide exchange rate in HLA-DRB1*03:01 monomer and theoretical peptide binding affinities to HLA-DRB1*03:01 FIG. 12 shows staining of CD4 lymphocytes with peptide exchanged HLA-DRB1*01:01 PE, APC and BV421 conjugated tetramers.

FIG. 13 shows staining of CD4 lymphocytes with peptide exchanged HLA-DRB1*04:01 PE conjugated tetramers.

FIG. 14 shows staining of CD4 lymphocytes with peptide exchanged HLA-DRB1*15:01 PE, APC and BV421 conjugated tetramers.

### DETAILED DESCRIPTION OF THE INVENTION

### General

Development of vaccines or T cell based immunotherapies often requires to test a large number of neoantigen peptides for MHC class II binding and T cell characterization. The system presented here allows to perform both tasks with dual use MHC class II recombinant complexes and does not require any catalyst or peptide exchange factor. On one hand the class II complex formed with a place holder peptide allows to measure quantitatively and non-quantitatively the affinity of the test peptide towards MHC class II by measuring the extent of its displacement. On the other hand, newly generated MHC class II/peptide monomers and tetramers can directly be used for assessing CD4+ T cell populations reacting specifically against neoantigens.

### Definitions

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The phrase “derived from” when used concerning a rearranged variable region gene “derived from” an unrearranged variable region and/or unrearranged variable region gene segments refers to the ability to trace the sequence of the rearranged variable region gene back to a set of unrearranged variable region gene segments that were rearranged to form a gene that expresses the variable domain (accounting for, where applicable, splice differences and somatic mutations). For example, a rearranged variable region gene that has undergone somatic mutation is still derived from the unrearranged variable region gene segments. In some embodiments, where the endogenous locus is replaced with a universal light chain or heavy chain locus, the term “derived from” indicates the ability to trace origin of the sequence to said rearranged locus even though the sequence may have undergone somatic mutations.

### MH Class II Proteins

The MHC class II proteins provided and used in the methods of the present disclosure may be any suitable MHC class II molecules known in the art where it is desirable to exchange the peptide that the MHC class II protein originally contained with another peptide. Generally, they have the formula (α-β-P)ₙ, where n is at least 2, for example between 2-10, e.g. 4. α is an α chain of a class II MHC protein. β is a β chain, herein defined as the β chain of a class II MHC protein. P is a peptide antigen.

The MHC class II proteins may be from any mammalian or avian species, e.g. primate sp., particularly humans; rodents, including mice, rats and hamsters; rabbits; equines, bovines, canines, felines; etc. For instance, the MHC class II protein may be derived the human HLA proteins or the murine H-2 proteins. HLA proteins include the class II subunits HLA-DPα, HLA-DPβ, HLA-DQα, HLA-DQβ, HLA-DRα and HLA-DRα. H-2 proteins include the class II subunits I-Aα, I-Aβ, I-Eα and I-Eβ, and β2-microglobulin. Sequences of some representative MHC class II proteins may be found in Kabat et al. Sequences of Proteins of Immunological Interest, NIH Publication No. 91-3242, pp 724-815. MHC class II protein subunits suitable for use in the present invention are a soluble form of the normally membrane-bound protein, which is prepared as known in the art, for instance by deletion of the transmembrane domain and the cytoplasmic domain. Soluble class II subunits will include the α1 and α2 domains for the a subunit, and the β1 and β2 domains for the R subunit.

The α and β subunits may be separately produced and allowed to associate in vitro to form a stable heteroduplex complex, or both of the subunits may be expressed in a single cell. Methods for producing MHC class II subunits are known in the art.

To prepare the MHC class II-peptide complex, the subunits may be combined with an antigenic peptide and allowed to fold in vitro to form a stable heterodimer complex with intrachain disulfide bonded domains. The peptide may be included in the initial folding reaction, or may be added to the empty heterodimer in a later step. In the methods of the present invention, this will be the exiting peptide. Conditions that permit folding and association of the subunits and peptide are known in the art. As one example, roughly equimolar amounts of solubilized α and β subunits may be mixed in a solution of urea. Refolding is initiated by dilution or dialysis into a buffered solution without urea. Peptides may be loaded into empty class II heterodimers at about pH 5 to 5.5 for about 1 to 3 days, followed by neutralization, concentration and buffer exchange. However, the specific folding conditions are not critical for the practice of the invention.

The monomeric complex (α-β-P) (herein monomer) may be multimerized. The resulting multimer will be stable over long periods of time. Preferably, the multimer may be formed by binding the monomers to a multivalent entity through specific attachment sites on the α or β subunit, as known in the art (e.g., as described in U.S. Pat. No. 5,635,363). The MHC class II proteins, in either their monomeric or multimeric forms, may also be conjugated to beads or any other support.

Frequently, the multimeric complex will be labeled, so as to be directly detectable when used in immunostaining or other methods known in the art, or will be used in conjunction with secondary labeled immunoreagents which will specifically bind the complex, as known in the art and as described herein. For example, the label may be a fluorophore, such as fluorescein isothiocyanate (FITC), rhodamine, Texas Red, phycoerythrin (PE), allophycocyanin (APC), Brilliant Violet™ 421, Brilliant UV™ 395, Brilliant Violet™ 480, Brilliant Violet™ 421 (BV421), Brilliant Blue™ 515, APC-R700, or APC-Fire750. In some embodiments, the multimeric complex is labeled by a moiety that is capable of specifically binding another moiety. For instance, the label may be biotin, streptavidin, an oligonucleotide, or a ligand. Other labels of interest may include dyes, enzymes, chemiluminescers, particles, radioisotopes, or other directly or indirectly detectable agent.

The methods disclosed herein may be used to perform peptide exchange on any suitable MHC class II protein. Exemplary MHC class II proteins of use in the methods and with the peptides disclosed here include HLA-DRB1*01:01 tetramer, HLA-DRB1*04:01 monomer, or HLA-DRB1*15:01 monomer. However, any MHC class II allele may be used in the methods herein upon selection of an appropriate exiting peptide, according for example to known techniques for predicting the affinity of a peptide to an MHC class II allele.

### Labels for Quantification of Peptide Exchange

According to some embodiments of the methods disclosed herein, the exiting peptide (i.e., the peptide that is to be exchanged) is labeled such that the proportion of the exiting peptide that remains bound to the MHC class II proteins may be quantified, for example by a sandwich immunoassay.

Labels particularly adapted to quantification of the exchange are those that do not render the peptide unable to bind to the MHC class II protein, and which are capable of specifically binding to a detecting moiety. Exemplary specific binding pairs include biotin or variants thereof with streptavidin or variants thereof, tags with their complementary antibodies, ligands, or lectins, haptens with the proteins they bind to, or oligonucleotide sequences with their complement.

Thus, in some embodiments an exiting peptide may be labeled with a tag, i.e. a moiety recognizable by an antibody, a lectin, or a specific ligand. Exemplary tags include dinitrophenol (DNP), sialic acid, nitrosyl, sulfated saccharides, 0-glycosylated amino acids, N-glycosylated amino acids, phosphoserine, phosphothreonine, and phosphotyrosine. For example, and without limitation, such labeled exiting peptides may include PK(DNP)PVSKMRMATPLLM, PK(DNP)PVSLMRMPTPLLM, K(DNP)PVSKMRMARPLLMQ, and/or PK(DNP)PVSKYRMATPLLM.

In some embodiments, an exiting peptide may be labeled with a biotin moiety or a known biotin variant for binding to streptavidin or a streptavidin variant. Exemplary biotin variants are 2-iminobiotin, carboxybiotin, biocytin, or iminobiocytin.

An exiting peptide labeled as described will bind with a binding partner specific to the label on the exiting peptide. The binding partner may be labeled with a moiety capable of direct detection, such as a fluorescent moiety or a radiolabel, a barcode, or a moiety capable of indirect detection, for instance an enzyme label such as horseradish peroxidase (HRP) or alkaline phosphatase. Then the MHC class II proteins that are unexchanged—i.e., those that retain the exiting peptide—may be detected, for instance, by a sandwich assay in which capture beads or any other suitable support conjugated with anti-MHC class II protein antibodies are used to capture the MHC class II protein, and the binding partner is used to identify those MHC class II proteins that are still bound to the exiting peptide.

In embodiments where the binding partner is labeled with an enzyme label (such as a HRP), instead of measuring fluorescence levels, a substrate of the enzyme (such as tetramethylbenzidine (TMB), 3,3′-diaminobenzidine (DAB), or 2,2′-azino-bis(3-ethylbenzothiazoline-6-sulphonic acid) (ABTS)) may be added to develop a detectable color. A high level of exchange leads to no exiting peptide remaining associated with the MHC class II proteins, and accordingly the anti-tag antibody does not bind, no enzyme is present, and no color develops. A low level of exchange leads to high retention of the exiting peptide, so a large amount of the anti-tag antibody binds, a large amount of the enzyme is present, so a color develops.

Exemplary fluorescent moieties that may be used to label the binding partner (e.g., that may be used to label the antibody) include 7-amino-4-methyl-coumarin (AMC), 5-((2-aminoethyl)amino)naphthalene-1-sulfonic acid (EDANS), FITC, FAM, 7-nitrobenz-2-oxa-1,3-diazole (NBD), Rhodamine B, TAMRA, or any other suitable fluorescent moiety.

Exemplary radiolabels that may be used to label the binding partner (e.g., that may be used to label the antibody) include ³²P, ³⁵S methionine, and ¹²⁵I. Methods for using such radiolabels in connection with antibodies, lectins, or ligands are known in the art.

### Exiting Peptides

Those of skill in the art will be able to create exiting peptides in addition to those described herein, including exiting peptides with various labels and various sequences that are adapted to bind at a suitable strength to any one of the many known MHC class II alleles. Such exiting peptides may be designed by known techniques, such as by available tools to predict the affinity of a particular peptide to a particular MHC class II allele. They may optionally be labeled as disclosed herein. Exemplary exiting peptides disclosed herein include PKPVSKMRMATPLLM, PKPVSLMRMATPLLM, KPVSKMRMARPLLMQ,

or PKPVSKYRMATPLLM. Other exemplary exiting peptides are those having at least 75%, 80%, 85%, 90%, 95%, 96%, 97%, 98%, or 99% sequence identity with the foregoing.

### Entering Peptides

Those of skill in the art will be able to create entering peptides in addition to those described herein, including entering peptides with various labels and various sequences that are adapted to bind at a suitable strength to any one of the many known MHC class II alleles, such as a binding strength that is greater or comparable to the exiting peptide being used. Such entering peptides may be designed by known techniques, such as by available tools to predict the affinity of a particular peptide to a particular MHC class II allele. They may optionally be labeled as disclosed herein. Exemplary entering peptides disclosed herein include TSLYNLRRGTALA, VSTIVPYIGPALNI, QYIKANSKFIGITE, TKIYSYFPSVISKV, PKYVKQNTLKLAT, or those included in Tables 1-3. Other exemplary entering peptides are those having at least 75%, 80%, 85%, 90%, 95%, 96%, 97%, 98%, or 99% sequence identity with the foregoing.

### Media for Exchange

The methods described herein may be performed in any suitable medium.

In some embodiments, the reaction mixture contains the reagents in a buffered solution. Thus, the reaction mixture may consist essentially of an MHC class II molecule bound to a first (exiting) peptide, and a second (entering) peptide. According to these embodiments, the MHC class II molecule, the first peptide, and the second binding partner (such as the second peptide) may all be as described herein.

In some embodiments, the reaction mixture contains the reagents in a solution, and the solution may comprise other species, such as peptides other than the first and second peptides, lipids, polynucleotides, or other biological or non-biological species. In certain embodiments, the solution may comprise bodily fluids, tissue extracts from normal or abnormal tissues, cell extracts from normal or abnormal tissues, cell extracts from tumors, or cell extracts from other pathologies. In certain embodiments, the solution may comprise extracts or lysates of microorganisms such as viruses, bacteria, parasites, or yeast. In certain embodiments, the solution may comprise environmental water, air and soil samples, or extracts thereof. In certain embodiments, the solution may comprise synthetic mixtures, such as protein hydrolysates, perfusions, vaccines, or synthetic tissue culture media. According to these embodiments, the MHC class II molecule, the first peptide, and the second binding partner (such as the second peptide) may all be as described herein.

### Kits

In some aspects, the present disclosure provides kits for peptide exchange, such as quantified peptide exchange. The kits may comprise several modules, which may include an MHC class II molecule module, a peptide detection module, and/or a reference peptide module. The components of the kit may be selected such that the user of the kit is able to prepare the remainder of the necessary components for conducting the methods disclosed herein.

The MHC class II molecule module will generally include the MHC class II protein for exchange (in monomeric or multimeric form), for instance including the exiting peptide lyophilized or in solution. The solution is advantageously buffered, and may also contain other components to stabilize the solution. For instance, the solution may contain protein stabilizers and/or sodium azide. The peptide associated with the MHC class II protein is generally labeled as described herein. The sequence of the peptide is selected to provide stability of the kit MHC class II protein and exchangeability against a peptide of interest.

The peptide detection module will generally contain any suitable peptide detection system that is matched with the labeled exchangeable peptide provided in the MHC class II module. For instance, if the detection system contemplates a sandwich magnetic bead immunoassay, then the detection module may contain a vial comprising magnetic beads coupled with MHC class II capture antibody, or streptavidin in case of peptide exchanged MHC class II biotinylated monomers, and a vial comprising a fluorescently-tagged antibody that is reactive against the exiting peptide (in the case of tagged peptides).

### EXAMPLES

The invention now being generally described, it will be more readily understood by reference to the following examples which are included merely for purposes of illustration of certain aspects and embodiments of the present invention, and are not intended to limit the invention.

### Example 1: Description of Peptide Exchange and Quantitation of Peptide Exchange on MHC Class II Monomers

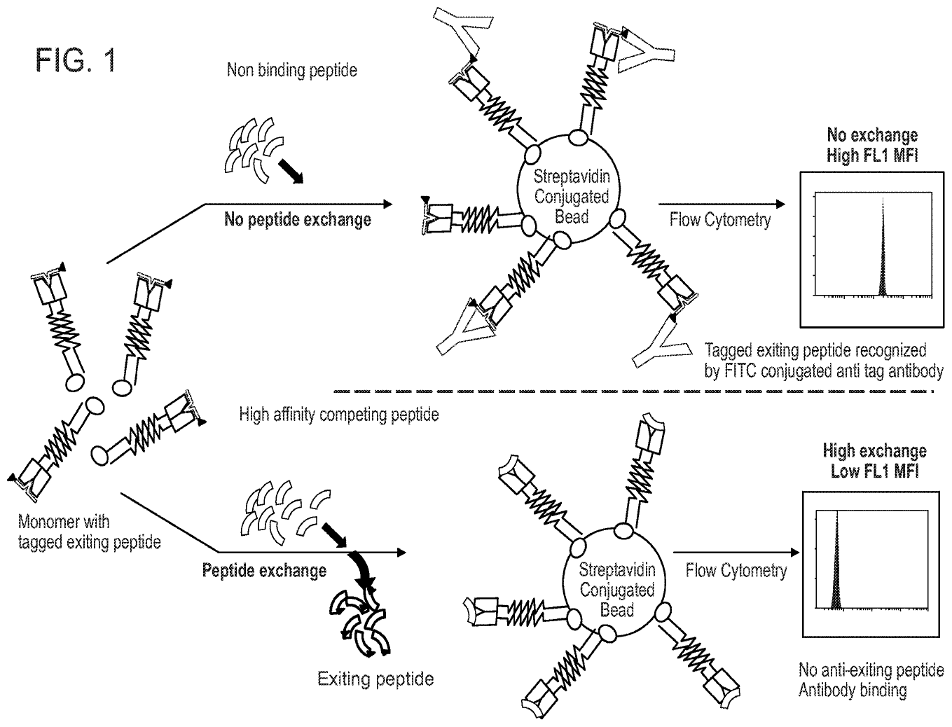

MHC class II monomers are formed by the combination of the extracellular domain of the MHC class II α chain and β chain to which leucine zipper motifs are added to force them to pair. The alpha chain is biotinylated at its C-terminus which allows MHC class II monomers to tetramerize by attachment to streptavidin. Bound to each MHC is the exiting peptide. For the exchange, entering peptide is added. The exiting peptide is displaced by entering peptide proportionally to its binding affinity to the MHC molecule. The resulting complex contains the entering peptide bound to the MHC monomers. If the entering peptide does not bind to the MHC, the exiting peptide is not displaced. Capturing monomers after reaction with streptavidin conjugated magnetic beads followed by staining with a fluorescent antibody recognizing the exiting peptide allows to quantitate the peptide exchange rate by flow cytometry. No exchange corresponds to a high MFI whereas 100% exchange corresponds to a MFI close to zero. Intermediary peptide exchange rates correspond to intermediary MFIs. A schematic diagram of peptide exchange and quantitation of peptide exchange on MHC class II monomers is shown in FIG. 1.

### Example 2: Exemplary Protocol for Measurement of Peptide Displacement in a Class II Biotinylated Monomer

Reagents: The following stock solutions are prepared or provided:

Biotinylated Monomer (e.g. HLA-DRB1*01:01) with exiting peptide in a buffered 100 μg/mL (measured by monomer content) solution with added protein stabilizers and <0.09% sodium azide.

Magnetic beads conjugated with streptavidin in a buffered NaCl 150 mM, Na₂HPO₄.2H₂O 6.5 mM, pH 7.1-7.35 solution with added protein stabilizers and <0.09% sodium azide.

Fluorescently tagged antibody (e.g. tagged with FITC) that is reactive against the exiting peptide, in a buffered NaCl 150 mM, Na₂HPO₄.2H₂O 6.5 mM, pH 7.1-7.35 solution with added protein stabilizers and 0.09% sodium azide.

Reference exchange peptide in 10 mM DMSO solution Desired exchange peptide in 10 mM DMSO-1 mM DMSO solution Peptide exchange solution: Na₂HPO₄.2H₂O 36 mM, sodium citrate 14.4 mM, NaN3 0.02% pH 5.5 (N-Dodecyl β-D-maltoside 0.1% (optional)). Store at 2-8° C.

Assay Buffer solution, NaCl 150 mM, Na₂HPO₄.2H₂O 6.5 mM, pH 7.1-7.35, with added protein stabilizers and 0.09% sodium azide (1.5 mL×1 vial with natural cap). Store at 2-8° C.

Peptide Exchange Procedure: 25 μL of the monomer solution is combined with 2.5 μL of the peptide solution and the resulting solution was mixed, then incubated overnight at 37° C.

Sandwich magnetic bead immunoassay: 20 μL streptavidin conjugated capture beads are pipetted into wells 1-4 on a 96-well plate. 2 μL assay buffer is pipetted into wells 1 and 2. 2 μL of non-exchanged monomer are pipetted into well 3. 2 μL of exchanged monomer are pipetted into well 4. Additional monomers may be pipetted into wells 5 and up. The plate is protected from light and shaken for 45 min at 550 rpms/min. 150 μL of assay buffer is dispensed into each well. The plate is placed on a plate magnet and the beads are permitted to sediment for at least 5 minutes.

Separately, a preparation of the FITC-conjugated anti-tag antibody is prepared at 1 μg/mL. 25 μL of the FITC-conjugated anti-tag antibody is pipetted into each well except well 1, and the plate is again protected from light and shaken for 45 min at 550 rpms/min.

150 μL of assay buffer is dispensed into each well, and the plate is placed on a plate magnet. The beads are permitted to sediment for at least 5 minutes. The beads are resuspended in sheath fluid and transferred to labeled flow cytometer tubes (referred to herein by the number of the well they are drawn from) or run directly on a plate format flow cytometer as described below.

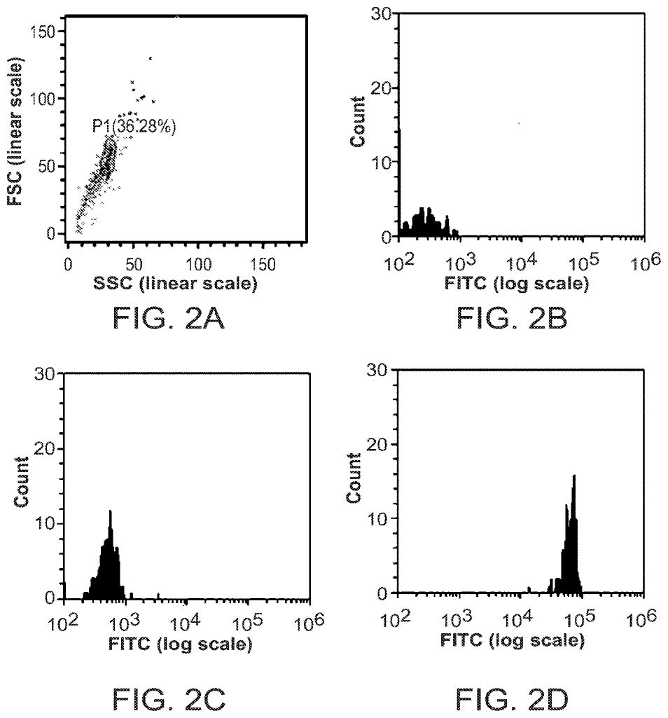

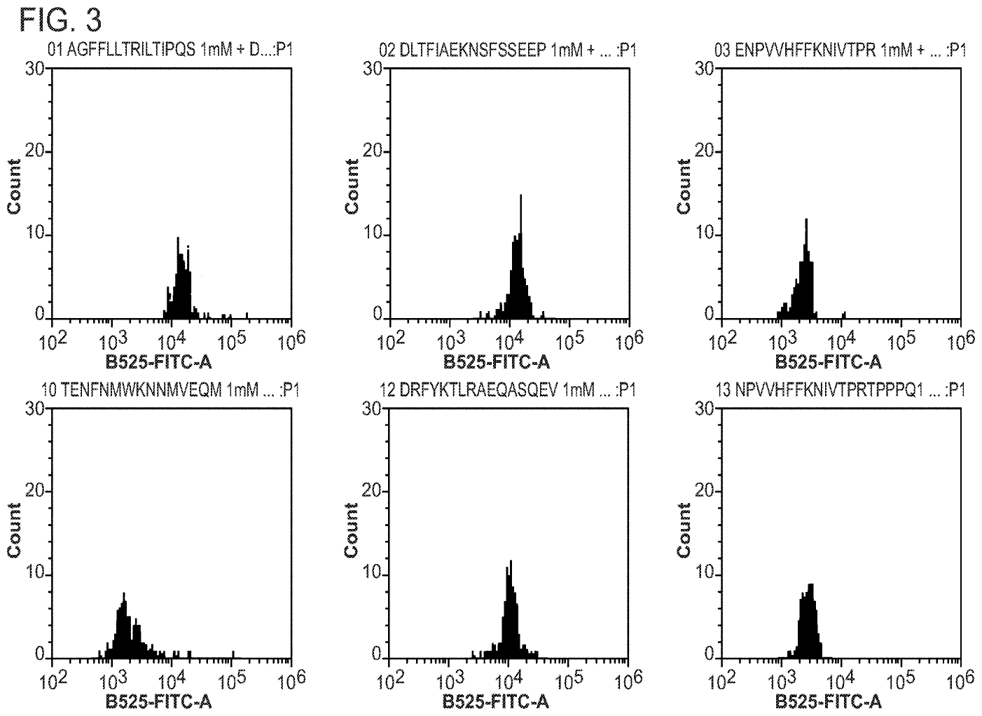

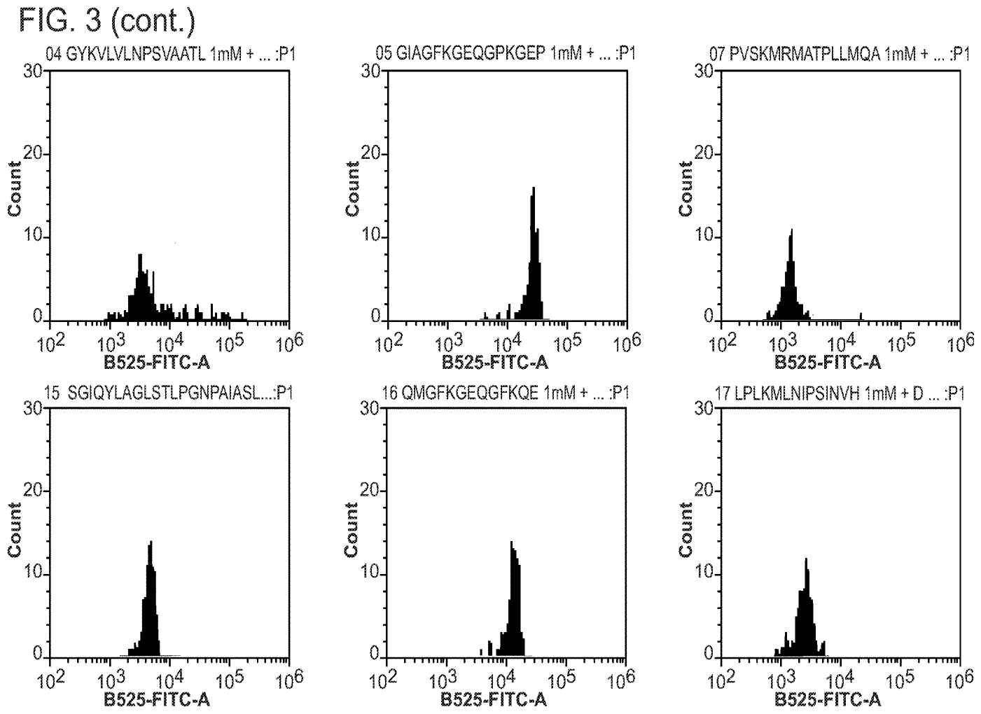

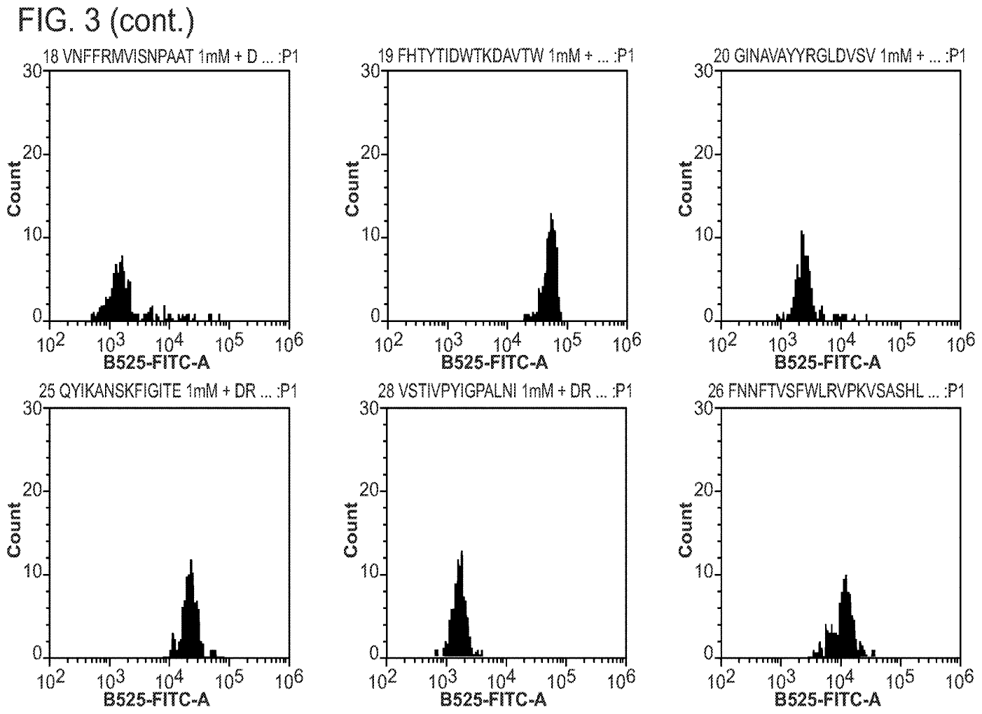

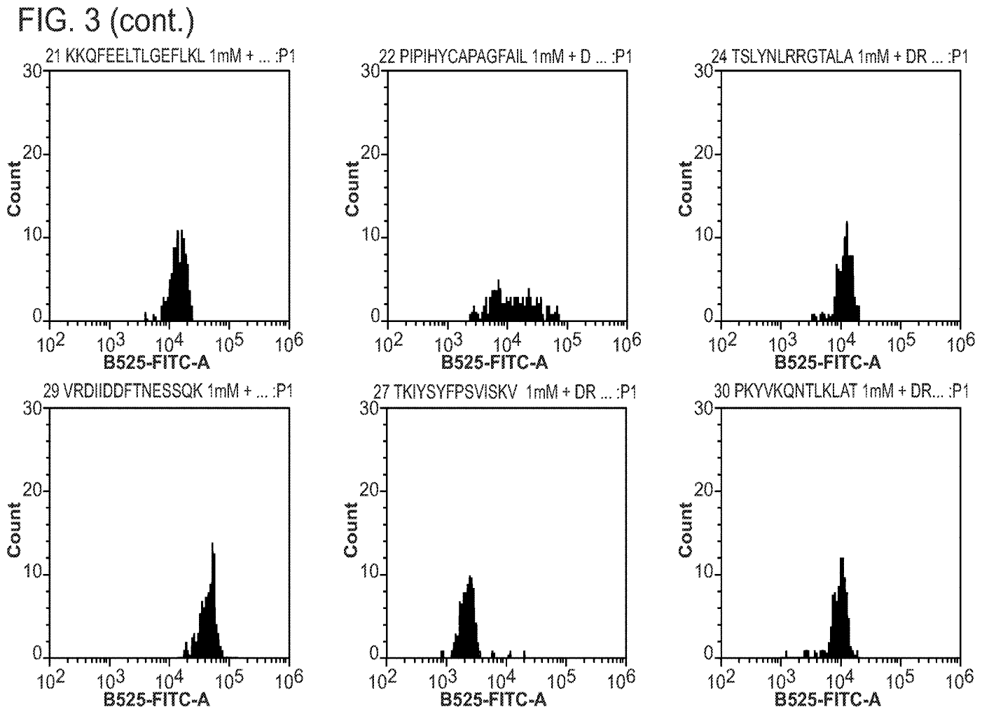

Flow Cytometry Analysis: Unused magnetic beads from well 1 are used to set FSC (forward-scattered light) and SSC (side-scattered light) voltages and gains such that a live gate selects single beads and excludes doublets and larger clumps, see FIG. 2A. PMT voltages and gains are set based on the unused beads to have mean fluorescence intensities (MFI) in the first log decade as seen in FIG. 2B. Well 2, which contains beads that have captured no monomer and therefore no tagged peptide, is run to calibrate the level for 0% tagged peptide, i.e. 100% peptide exchange (see FIG. 2C). Well 3, which contains a solution where all monomers display the original tagged peptide, is run to calibrate the level for 100% tagged peptide, i.e. 0% peptide exchange (see FIG. 2D). Well 4 and up are run to evaluate the samples. Peptide exchanged monomers will display various quantities of tagged peptides, depending on their binding affinities to MHC class II molecules. Therefore, the FL1 MFI of the different test peptide exchanged monomers measured on the flow cytometer will be intermediary between MFI values obtained with wells 2 and 3 (see FIG. 3). The control data can then be interpolated to find the percent of tagged peptide in the different test samples.

### Example 3: Description of Peptide Exchange and Quantitation of Peptide Exchange on MHC Class II Tetramers

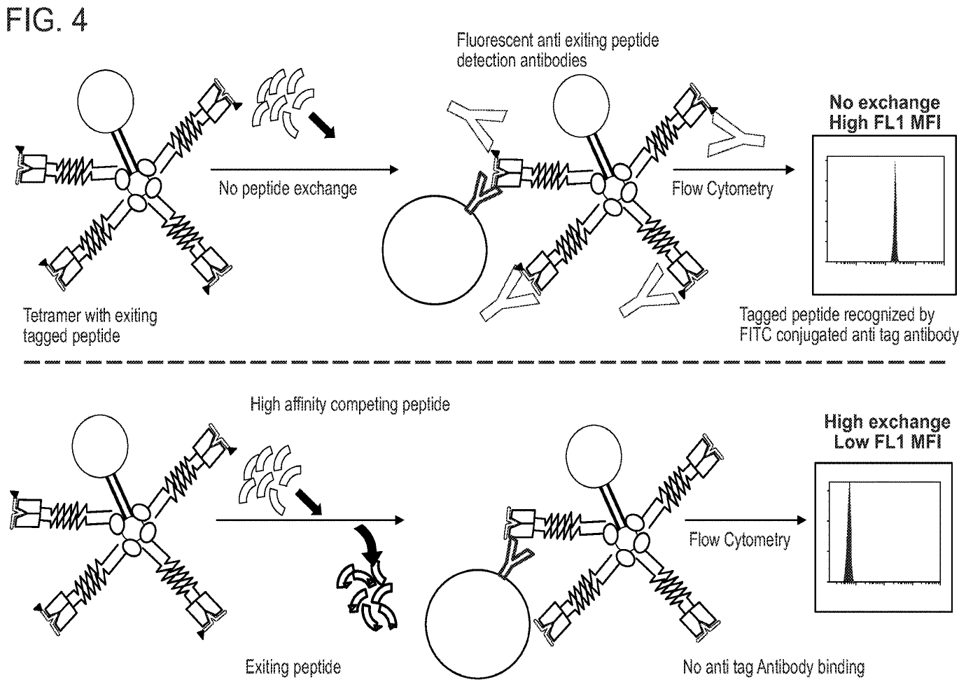

MHC class II tetramers are formed by the binding of four biotinylated MHC class II monomers per streptavidin molecule. The tetrameric MHC complex is labeled with a fluorophore. Bound to each MHC monomer in the complex is the exiting peptide. For the exchange, entering peptide is added. The exiting peptide is displaced by entering peptide proportionally to its binding affinity to the MHC molecule. The resulting complex contains the entering peptide bound to the MHC monomers. If the entering peptide does not bind to the MHC, the exiting peptide is not displaced. Capturing tetramers after reaction with magnetic beads followed by staining with a fluorescent antibody recognizing the exiting peptide allows to quantitate the peptide exchange rate by flow cytometry. No exchange corresponds to a high MFI whereas 100% exchange corresponds to a MFI close to zero. Intermediary peptide exchange rates corresponds to intermediary MFIs. A schematic diagram of peptide exchange and quantitation of peptide exchange on MHC class II tetramers is shown in FIG. 4.

### Example 4: Exemplary Protocol for Measurement of Peptide Displacement in a Class II Tetramer

Reagents: the following stock solutions are prepared or provided:

Tetramer (e.g. HLA-DRB1*01:01) with exiting peptide in a buffered 50 μg/mL (measured by monomer content) solution with added protein stabilizers and <0.09% sodium azide.

Magnetic beads conjugated with a tetramer capture antibody in a buffered NaCl 150 mM, Na₂HPO₄.2H₂O 6.5 mM, pH 7.1-7.35 solution with added protein stabilizers and <0.09% sodium azide.

Fluorescently tagged antibody (e.g. tagged with FITC) that is reactive against the exiting peptide, in a buffered NaCl 150 mM, Na₂HPO₄.2H₂O 6.5 mM, pH 7.1-7.35 solution with added protein stabilizers and 0.09% sodium azide.

Reference exchange peptide in 10 mM DMSO solution

Desired exchange peptide in 10 mM DMSO-1 mM DMSO solution

Assay Buffer solution, NaCl 150 mM, Na₂HPO₄.2H₂O 6.5 mM, pH 7.1-7.35, with (N-Dodecyl β-D-maltoside 0.1% (optional)) added protein stabilizers and <0.09% sodium azide (1.5 mL×1 vial with natural cap). Store at 2-8° C.

Peptide Exchange Procedure: 50 μL of the tetramer solution is combined with 5 μL of the peptide solution and the resulting solution was mixed, then incubated overnight at 37° C.

Sandwich magnetic bead immunoassay: 20 μL capture beads are pipetted into wells 1-4 on a 96-well plate. 5 μL assay buffer is pipetted into well 2. 5 μL of non-exchanged tetramer are pipetted into wells 1 and 3. 5 μL of exchanged tetramer are pipetted into well 4. Additional exchanged tetramers may be pipetted into wells 5 and up. The plate is protected from light and shaken for 45 min at 550 rpms/min. 150 μL of assay buffer is dispensed into each well. The plate is placed on a plate magnet and the beads are permitted to sediment for at least 5 minutes.

Separately, a preparation of the FITC-conjugated anti-tag antibody is prepared at 10 μg/mL. 25 μL of the FITC-conjugated anti-tag antibody is pipetted into each well except wells 1 and 2, and the plate is again protected from light and shaken for 45 min at 550 rpms/min.

150 μL of assay buffer is dispensed into each well, and the plate is placed on a plate magnet. The beads are permitted to sediment for at least 5 minutes. The beads are resuspended in sheath fluid and transferred to labeled flow cytometer tubes or analyzed directly on a plate format flow cytometer, as described below.

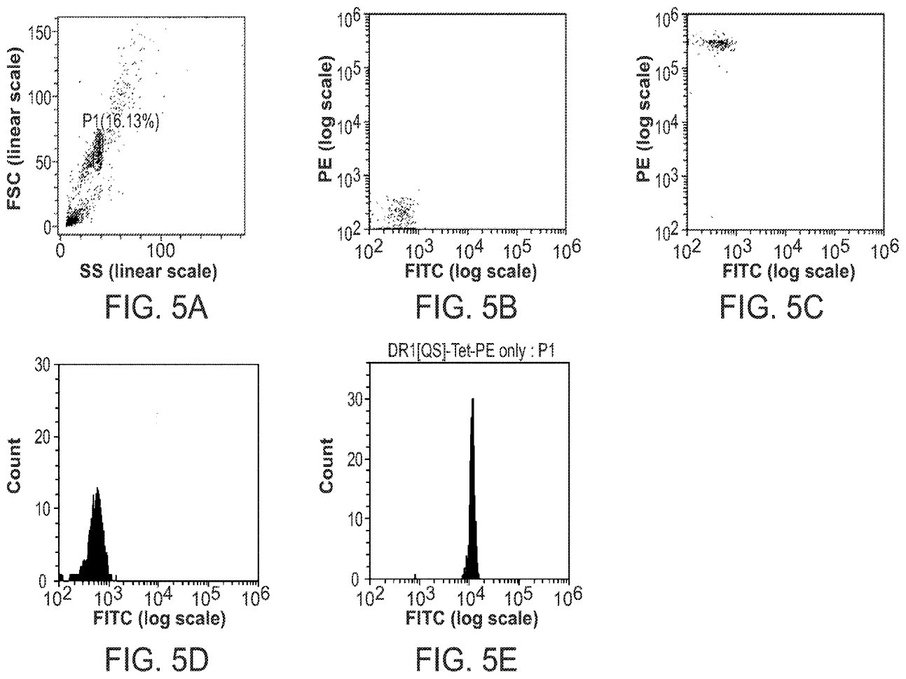

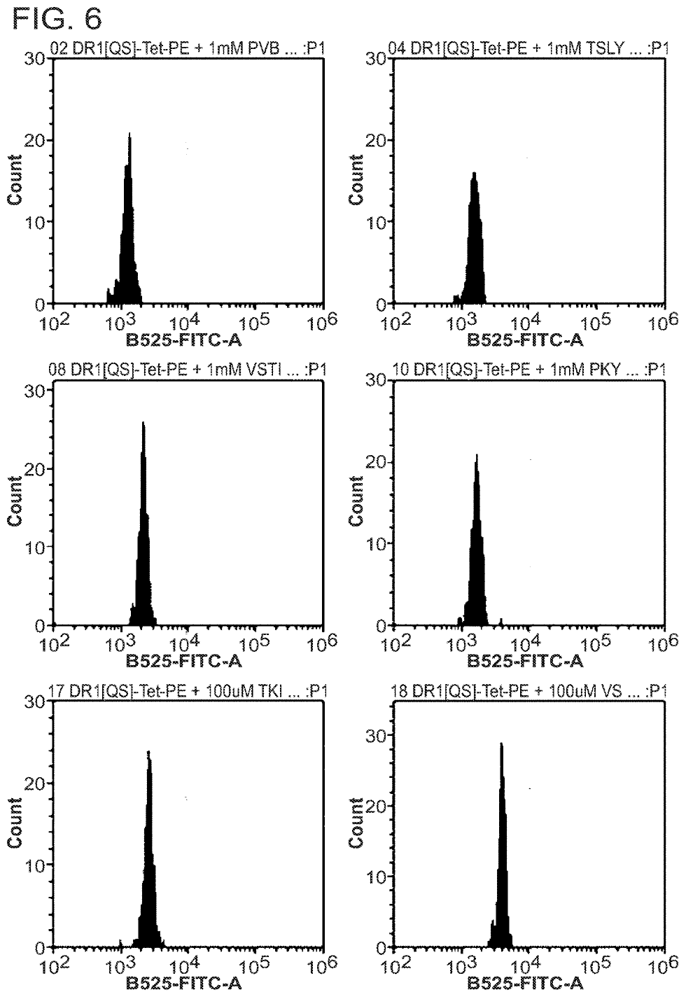

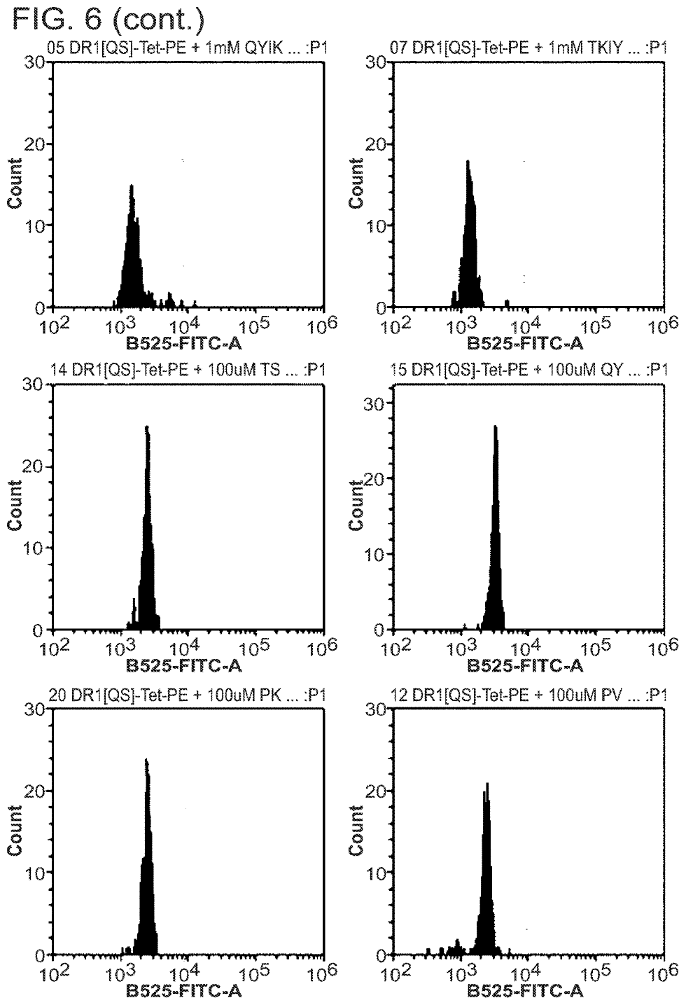

Flow Cytometry Analysis: Unused magnetic beads are run to set FSC (forward-scattered light) and SSC (side-scattered light) voltages and gains such that a live gate selects single beads and excludes doublets and larger clumps, see FIG. 5A. PMT voltages and gains are set based on the unused beads to have mean fluorescence intensities (MFI) in the first log decade as seen in FIG. 5B. Well 1 is run to perform compensation in FL1 in order to eliminate fluorochrome interference in the FITC channel, see FIG. 5C. The FL1 MFI is set to the first log decade, close to that obtained with beads only. Well 2, which contains beads that have captured no tetramer and therefore no tagged peptide, is run to calibrate the level for 0% tagged peptide, i.e. 100% peptide exchange (see FIG. 5D). Well 3, which contains a solution where all tetramers display the original tagged peptide, is run to calibrate the level for 100% tagged peptide, i.e. 0% peptide exchange (see FIG. 5E). Wells 4 and up are run to evaluate the samples. Peptide exchanged tetramers will display various quantities of tagged peptides, depending on their binding affinities to MHC class II molecules. Therefore, the FL1 MFI measured on the test wells will be intermediary between MFI values obtained with wells 2 and 3 (see FIG. 6). The control data can then be interpolated to find the percent of tagged peptide in the sample.

### Example 5: HLA-DRB1*01:01 Tetramers Generated by Peptide Exchange Display Various Staining Patterns that Depend on the Peptides Used for Exchange

50 μL of HLA-DRB1*01:01 Tetramer with exiting peptide PK(DNP)PVSKMRMATPLLM was mixed with 5 μL of 10 mM or 1 mM peptides dissolved in 10% DMSO. These peptides were respectively PVSKMRMATPLLMQA (CLIP, negative control), TSLYNLRRGTALA, QYIKANSKFIGITE, TKIYSYFPSVISKV, VSTIVPYIGPALNI (all 4 Tetanus Toxin Peptides) and PKYVKQNTLAT (Flu Peptide). The mixtures were incubated at 37° C. overnight, protected from light. PBMCs stimulated with all peptides (except PVSKMRMATPLLMQA) for 15 days in presence of 10% AB serum) were resuspended at 5×10⁶ cells per mL in PBS containing 0.2% BSA and 0.1% NaN3. 100 μL of cells were incubated for 120 minutes at 37° C. with 10 μL of tetramers obtained by peptide exchange and 50 mM Dasatinib. Cells were then stained with anti CD3 and anti CD4 mAbs for 20 min at RT. After a wash with PBS containing 0.2% BSA and 0.1% NaN3, cells were resuspended in 200 μL of PBS containing 0.5% formaldehyde and then were analyzed on a Fortessa flow cytometer. Stainings were performed with freshly exchanged tetramers or tetramers that had been exchanged 2 months prior. Among these tetramers, two different peptide final concentrations were used: 100 uM and 1 mM.

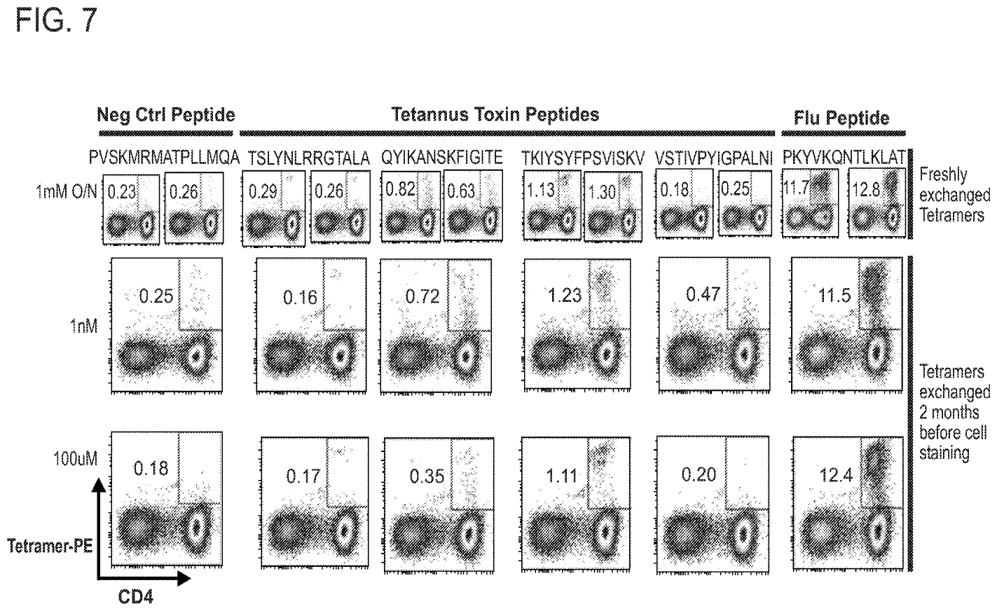

Results are shown in FIG. 7. Results indicate that exchanged tetramers with the negative control peptide were virtually negative. Tetramers exchanged with two Tetanus Toxin peptides (TSLYNLRRGTALA and VSTIVPYIGPALNI) gave similar staining patterns as the negative control tetramer indicating that the patient did not respond to these peptides. Two other Tetanus toxin peptides (QYIKANSKFIGITE and TKIYSYFPSVISKV) generated tetramers that stained patient's cells with different staining patterns, indicating that the TCR reacting against them were different in terms of number and/or affinity. The tetramer exchanged with the Flu peptide PKYVKQNTLKLAT stained a large percentage of CD4+ T cells, indicating that the donor had a strong immunity against that antigen. Comparison of positivity percentages as well as staining patterns indicated that peptide exchanged tetramers are stable and share the same activity as the ones that are freshly prepared. Finally, similar patterns and percentage of positive cells duplicate stainings showed that tetramers are stable and reliable staining agents.

### Example 6: Comparison of Peptide Exchange Rates in HLA-DRB1*01:01 Tetramers and Theoretical Binding Affinity of Tested Peptides Towards HLA-DRB1*01:01

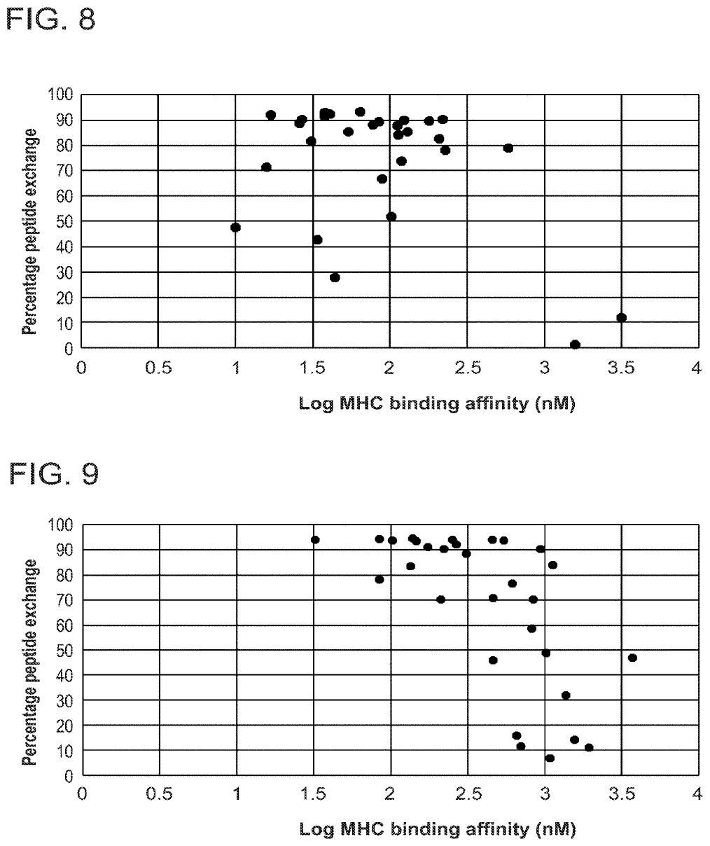

The IEDB stabilization matrix alignment method SMM-align was used to determine the theoretical binding affinities of the tested peptides towards HLA-DRB1*01:01 (Nielsen M et al. (2007) BMC Bioinformatics 8:238). Results (see Table 1) show a big spread of exchange rates for peptides in the 32-100 nm affinity range. See FIG. 8.

TABLE 1
Exchange rates for peptides in the 32-100 nm affinity range

| peptide DRB1*01:01 binding | % pept exch | smm_align_ic50 | log smm |
|---|---|---|---|
| AGFFLLTRILTIPQS | 88.70474499 | 26 | 1.414973 |
| AKFVAAWTLKAAA | 27.90061867 | 44 | 1.643453 |
| DLTFIAEKNSFSSEEP | 85.31757776 | 129 | 2.11059 |
| DRFYKTLRAEQASQEV | 81.5935963 | 31 | 1.491362 |
| ENPVVHFFKNIVTPR | 78.14583069 | 229 | 2.359835 |
| FHTYTIDWTKDAVTVV | 1.118956017 | 1572 | 3.196453 |
| FNNFIVSFWLRVPKVSASHLE | 89.53268789 | 180 | 2.255273 |
| GIAGFKGEQGPKGE | 82.49834411 | 209 | 2.320146 |
| GIAGFKGEQGPKGEP | 90.32751307 | 221 | 2.344392 |
| GINAVAYYRGLDVSV | 84.13379557 | 113 | 2.053078 |
| GYKVLVLNPSVAATL | 71.22211418 | 16 | 1.20412 |
| KKQFEELTLGEFLKL | 79.04846461 | 588 | 2.769377 |
| KRWIILGLNKIVRMY | 85.41622627 | 54 | 1.732394 |
| LPLKMLNIPSINVH | 42.74087854 | 34 | 1.531479 |
| NPVVHFFKNIVTPRTPPPQ | 89.40726335 | 85 | 1.929419 |
| PIPIHYCAPAGFAIL | 88.25378035 | 77 | 1.886491 |
| PKYVKQNTLKLAT | 91.55357319 | 38 | 1.579784 |
| PVSKMRMATPLLMQA | 91.98339886 | 17 | 1.230449 |
| QYIKANSKFIGITE | 73.87787314 | 118 | 2.071882 |
| RELERFAVNPGLLET | 66.67018983 | 89 | 1.94939 |
| SGIQYLAGLSTLPGNPAIASL | 90.36979101 | 27 | 1.431364 |
| SLQPLALEGSLQSRG | 89.90402909 | 123 | 2.089905 |
| TENFNMWKNNMVEQM | 51.69886836 | 102 | 2.0086 |
| TKIYSYFPSVISKV | 92.49355261 | 41 | 1.612784 |
| TSLYNLRRGTALA | 92.84798264 | 38 | 1.579784 |
| VNFFRMVISNPAAT | 47.53801491 | 10 | 1 |
| VRDIIDDFTNESSQK | 11.81104582 | 3148 | 3.498035 |
| VSTIVPYIGPALNI | 87.73728491 | 111 | 2.045323 |
| YALFYKLDVVPIDND | 93.21720994 | 64 | 1.80618 |

### Example 7: Comparison of Peptide Exchange Rates in HLA-DRB1*04:01 Monomers and Theoretical Binding Affinity of Tested Peptides Towards HLA-DRB1*04:01

The IEDB stabilization matrix alignment method SMM-align was used to determine the theoretical binding affinities of the tested peptides towards HLA-DRB1*04:01 (Nielsen M et al. (2007) BMC Bioinformatics 8:238). Results (see Table 2) show a big spread of exchange rates for peptides in the 300-3000 nm affinity range. See FIG. 9.

TABLE 2
Exchange rates for peptides in the 300-3000 nm affinity range

| peptide DRB1*04:01 binding | % pept exch | smm_align_ic50 | log smm |
|---|---|---|---|
| AGFFLLTRILTIPQS | 78.22186863 | 84 | 1.92427929 |
| AKFVAAWTLKAAA | 83.27403376 | 134 | 2.1271048 |
| DLTFIAEKNSFSSEEP | 90.94737209 | 173 | 2.2380461 |
| DRFYKTLRAEQASQEV | 93.77013742 | 541 | 2.73319727 |
| ENPVVHFFKNIVTPR | 45.82686849 | 457 | 2.6599162 |
| FHTYTIDWTKDAVTVV | 48.8071673 | 1024 | 3.01029996 |
| FNNFTVSFWLRVPKVSASHLE | 6.826556277 | 1077 | 3.0322157 |
| GIAGFKGEQGPKGE | 58.51924872 | 823 | 2.91539984 |
| GIAGFKGEQGPKGEP | 70.07117534 | 838 | 2.92324402 |
| GINAVAYYRGLDVSV | 11.49407982 | 703 | 2.84695533 |
| GYKVLVLNPSVAATL | 94.54892968 | 138 | 2.13987909 |
| KKQFEELTLGEFLKL | 46.93673462 | 3688 | 3.56679091 |
| KRWIILGLNKIVRMY | 76.62466302 | 615 | 2.78887512 |
| LPLKMLNIPSINVH | 70.16975262 | 211 | 2.32428246 |
| NPVVHFFKNIVTPRTPPPQ | 93.43890506 | 146 | 2.16435286 |
| PIPIHYCAPAGFAIL | 11.06379472 | 1933 | 3.28623185 |
| PKYVKQNTLKLAT | 93.667915 | 102 | 2.00860017 |
| PVSKMRMATPLLMQA | 92.21920926 | 265 | 2.42324587 |
| QYIKANSKFIGITE | 31.97105441 | 1365 | 3.13513265 |
| RELERFAVNPGLLET | 90.18379434 | 940 | 2.97312785 |
| SGIQYLAGLSTLPGNPAIASL | 93.83289724 | 251 | 2.39967372 |
| SLQPLALEGSLQSRG | 93.86522805 | 453 | 2.6560982 |
| TENFNMWKNNMVEQM | 83.99941678 | 1125 | 3.05115252 |
| TKIYSYFPSVISKV | 94.30422979 | 84 | 1.92427929 |
| TSLYNLRRGTALA | 14.12175087 | 1552 | 3.19089172 |
| VNFFRMVISNPAAT | 93.85873019 | 32 | 1.50514998 |
| VRDIIDDFTNESSQK | 88.52065923 | 308 | 2.48855072 |
| VSTIVPYIGPALNI | 15.84178187 | 656 | 2.81690384 |
| WITQCFLPVFLAQPPSGQRR | 70.64964333 | 460 | 2.66275783 |
| YALFYKLDVVPIDND | 90.24687112 | 221 | 2.34439227 |

### Example 8: Comparison of Peptide Exchange Rates in HLA-DRB1*15:01 Monomers and Theoretical Binding Affinity of Tested Peptides Towards HLA-DRB1*15:01

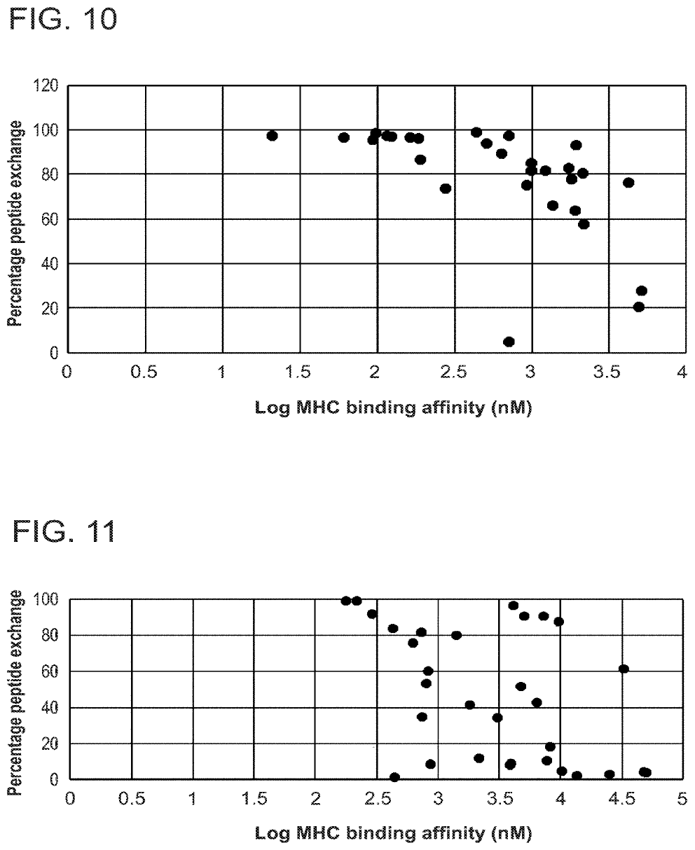

The IEDB stabilization matrix alignment method SMM-align was used to determine the theoretical binding affinities of the tested peptides towards HLA-DRB1*15:01 (Nielsen M et al. (2007) BMC Bioinformatics 8:238). Results (see Table 3) show a big spread of exchange rates for peptides in the 1000-5000 nm affinity range. See FIG. 10.

TABLE 3
Exchange rates for peptides in the 1000-5000 nm affinity range

| peptide DRB1*15:01 binding | % pept exch | smm_align_ic50 | log smm |
|---|---|---|---|
| AGFFLLTRILTIPQS | 73.58195203 | 278 | 2.444045 |
| AKFVAAVVTLKAAA | 5.050975419 | 708 | 2.850033 |
| DLTFIAEKNSFSSEEP | 78.02062795 | 1809 | 3.257439 |
| DRFYKTLRAEQASQEV | 82.99019462 | 1734 | 3.239049 |
| ENPVVHFFKNIVTPR | 97.1703441 | 21 | 1.322219 |
| FHTYTIDWTKDAVTW | 20.72290311 | 4912 | 3.691258 |
| FNNFTVSFWLRVPKVSASHLE | 81.50634262 | 1222 | 3.087071 |
| GIAGFKGEQGPKGE | 80.60060834 | 2126 | 3.327563 |
| GIAGFKGEQGPKGEP | 57.77841628 | 2148 | 3.332034 |
| GINAVAYYRGLDVSV | 96.12474529 | 185 | 2.267172 |
| GYKVLVLNPSVAATL | 86.54295179 | 189 | 2.276462 |
| KKQFEELTLGEFLKL | 76.25589099 | 4205 | 3.623766 |
| LPLKMLNIPSINVH | 97.09669642 | 123 | 2.089905 |
| NPVVHFFKNIVTPRTPPPQ | 96.43452788 | 61 | 1.78533 |
| PIPIHYCAPAGFAIL | 75.16900985 | 924 | 2.965672 |
| PKYVKQNTLKLAT | 85.11573914 | 993 | 2.996949 |
| PVSKMRMATPLLMQA | 98.73114636 | 438 | 2.641474 |
| QYIKANSKFIGITE | 66.14287649 | 1357 | 3.13258 |
| RELERFAVNPGLLET | 97.19560888 | 715 | 2.854306 |
| SGIQYLAGLSTLPGNPAIASL | 93.76521254 | 512 | 2.70927 |
| SLQPLALEGSLQSRG | 93.1070071 | 1928 | 3.285107 |
| TENFNMWKNNMVEQM | 96.39324555 | 164 | 2.214844 |
| TKIYSYFPSVISKV | 97.29798906 | 115 | 2.060698 |
| TSLYNLRRGTALA | 81.62771266 | 987 | 2.994317 |
| VNFFRMVISNPAAT | 95.58014881 | 94 | 1.973128 |
| VRDIIDDFTNESSQK | 27.65222446 | 5091 | 3.706803 |
| VSTIVPYIGPALNI | 98.4051811 | 98 | 1.991226 |
| WITQCFLPVFLAQPPSGQRR | 63.90586969 | 1898 | 3.278296 |
| YALFYKLDVVPIDND | 89.25734743 | 639 | 2.805501 |

### Example 9: Comparison of Peptide Exchange Rates in HLA-DRB1*03:01 Monomers and Theoretical Binding Affinity of Tested Peptides Towards HLA-DRB1*03:01

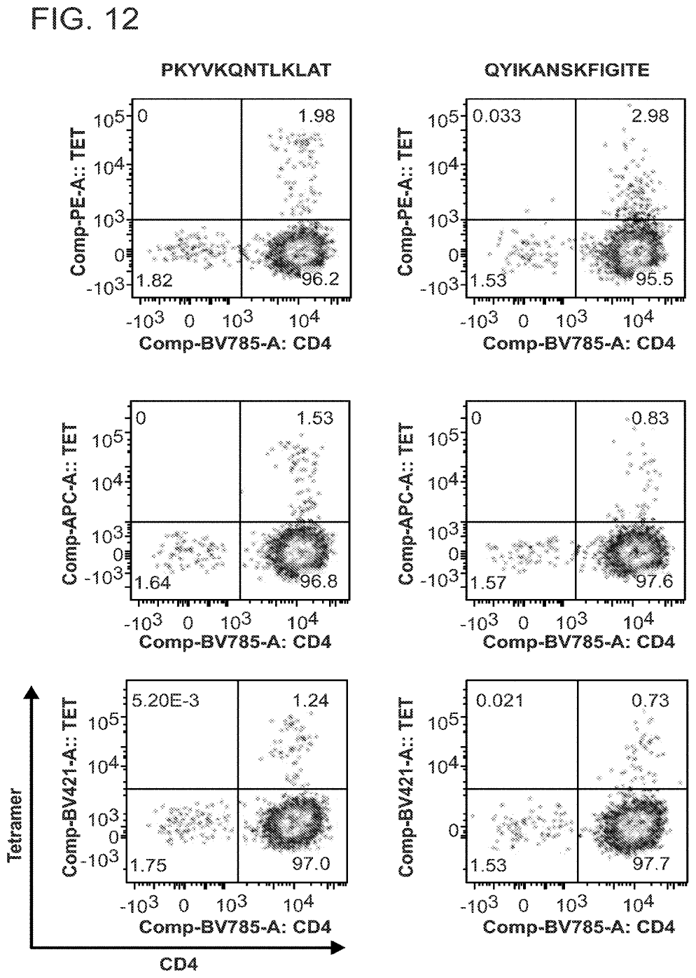

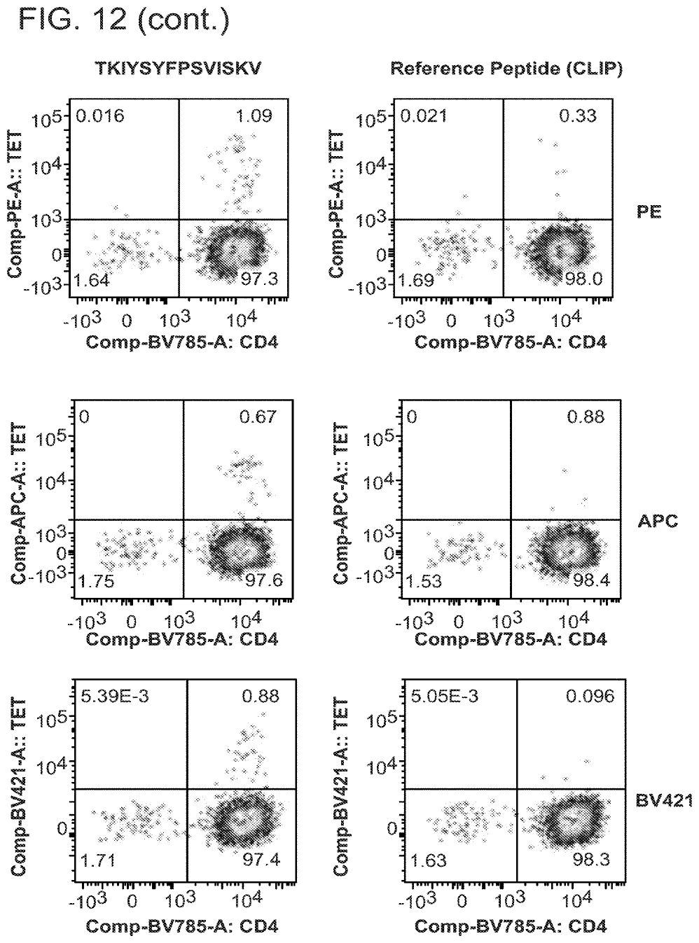

The IEDB stabilization matrix alignment method SMM-align was used to determine the theoretical binding affinities of the tested peptides towards HLA-DRB1*03:0101 (Nielsen M et al. (2007) BMC Bioinformatics 8:238). Results (see Table 4) show a big spread of exchange rates for peptides in the 300-13500 nm affinity range. See FIG. 11.

TABLE 4
Exchange rates for peptides in the 300-13500 nm affinity range.

| peptide DRB1*03:01 binding | % pept exch | smm_align_ic50 | log smm |
|---|---|---|---|
| AGFFLLTRILTIPQS | 83.59177635 | 2.629409599 | 83.59177635 |
| AKFVAAWTLKAAA | 34.70175208 | 2.868056362 | 34.70175208 |
| DLTFIAEKNSFSSEEP | 96.31084305 | 3.618152733 | 96.31084305 |
| DRFYKTLRAEQASQEV | 10.8993545 | 3.886547123 | 10.8993545 |
| ENPVVHFFKNIVTPR | 1.445302145 | 2.645422269 | 1.445302145 |
| FHTYTIDWTKDAVTW | 8.448073119 | 2.939019776 | 8.448073119 |
| FNNFIVSFWLRVPKVSASHLE | 42.66802006 | 3.805568818 | 42.66802006 |
| GIAGFKGEQGPKGE | 4.448399383 | 4.679282502 | 4.448399383 |
| GIAGFKGEQGPKGEP | 4.152949626 | 4.698926573 | 4.152949626 |
| GINAVAYYRGLDVSV | 90.59126203 | 3.860158261 | 90.59126203 |
| GYKVLVLNPSVAATL | 81.61958125 | 2.861534411 | 81.61958125 |
| ISNQLTLDSNTKYFHKLN | 98.85581191 | 2.336459734 | 98.85581191 |
| KKQFEELTLGEFLKL | 8.147072351 | 3.588159616 | 8.147072351 |
| KRWIILGLNKIVRMY | 75.59231559 | 2.797267541 | 75.59231559 |
| LPLKMLNIPSINVH | 18.19417665 | 3.918082785 | 18.19417665 |
| NPVVHFFKNIVTPRTPPPQ | 79.89072437 | 3.151063253 | 79.89072437 |
| PIPIHYCAPAGFAIL | 61.26141639 | 4.515926803 | 61.26141639 |
| PKYVKQNTLKLAT | 41.71449222 | 3.26245109 | 41.71449222 |
| PVSKMRMATPLLMQA | 99.02653384 | 2.247973266 | 99.02653384 |
| QYIKANSKFIGITE | 90.49904727 | 3.700790221 | 90.49904727 |
| RELERFAVNPGLLET | 11.95676555 | 3.336259552 | 11.95676555 |
| SGIQYLAGLSTLPGNPAIASL | 3.203386797 | 4.402261382 | 3.203386797 |
| SLQPLALEGSLQSRG | 34.41559177 | 3.482873584 | 34.41559177 |
| TENFNMWKNNMVEQM | 51.8519987 | 3.678336247 | 51.8519987 |
| TKIYSYFPSVISKV | 8.871989143 | 3.591621038 | 8.871989143 |
| TSLYNLRRGTALA | 2.404154422 | 4.131265697 | 2.404154422 |
| VNFFRMVISNPAAT | 53.25311933 | 2.906873535 | 53.25311933 |
| VRDIIDDFTNESSQK | 91.77645963 | 2.459392488 | 91.77645963 |
| VSTIVPYIGPALNI | 4.987527217 | 4.014268457 | 4.987527217 |
| WITQCFLPVFLAQPPSGQRR | 87.65840208 | 3.984662306 | 87.65840208 |
| YALFYKLDVVPIDND | 60.2317604 | 2.920645001 | 60.2317604 |

### Example 10: Staining of CD4 Lymphocytes with Peptide Exchanged HLA-DRB1*01:01 PE, APC and BV421 Conjugated Tetramers

25 μL of HLA-DRB1*01:01 monomer with exiting peptide PK(DNP)PVSKMRMATPLLM was mixed with 2 μL of emulsifier and 3 μL of 10 mM peptides dissolved in 10% DMSO. These peptides were respectively PKYVKQNTLAT (Flu Peptide), QYIKANSKFIGITE, TKIYSYFPSVISKV, (2 Tetanus Toxin Peptides) and PVSKMRMATPLLMQA (CLIP, negative control). Each peptide exchange monomer (30 μL) was mixed with 10 μL streptavidin-PE, streptavidin-APC or streptavidin-BV421. The tetramers were then mixed with 10 μL Neutralizer.

PBMCs from a donor expressing HLA-DRB1*01:01 were cultured at 3×10⁵ cells per well and stimulated with peptides. Before staining, cells with incubated with a 10% human AB serum block. Cells were treated with 50 nM Dasatinib and stained with tetramers for 2 hours at 37 C followed by a 20 min staining with anti CD3, CD4 & CD8 antibodies (anti CD3 FITC, anti CD8-BV510, anti CD4 BV785). Note that the percentages of tetramer positive cells are not significantly affected by the type of fluorochrome conjugated to streptavidin. See FIG. 12.

### Example 11: Staining of CD4 Lymphocytes with Peptide Exchanged HLA-DRB1*04:01 PE Conjugated Tetramers. High Affinity Peptides to MHC can Exchange with Final Concentrations as Low as 10 μM

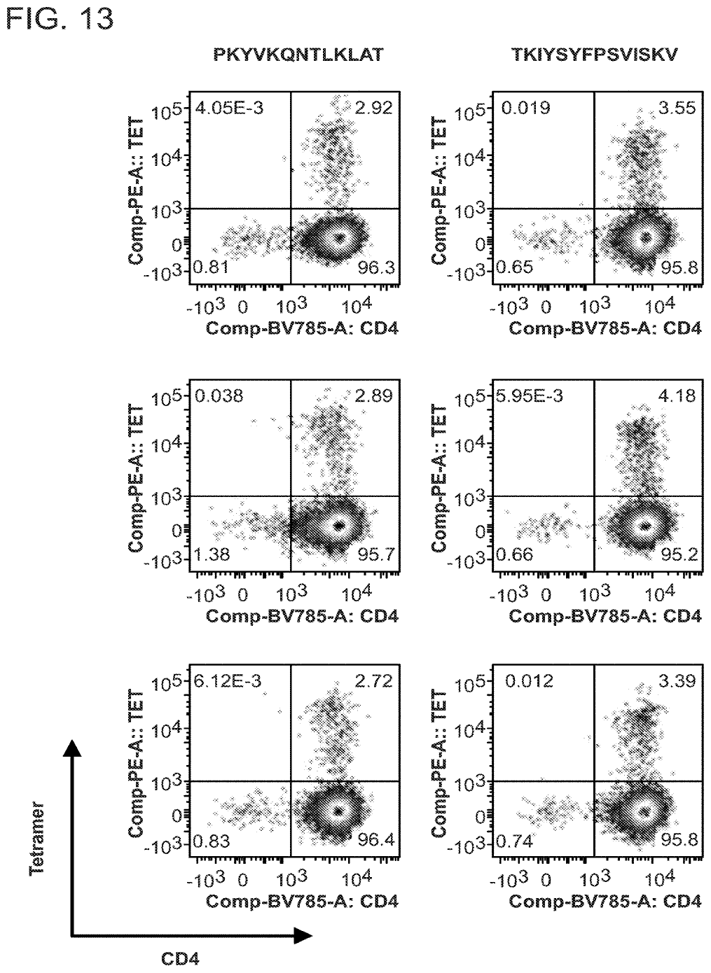

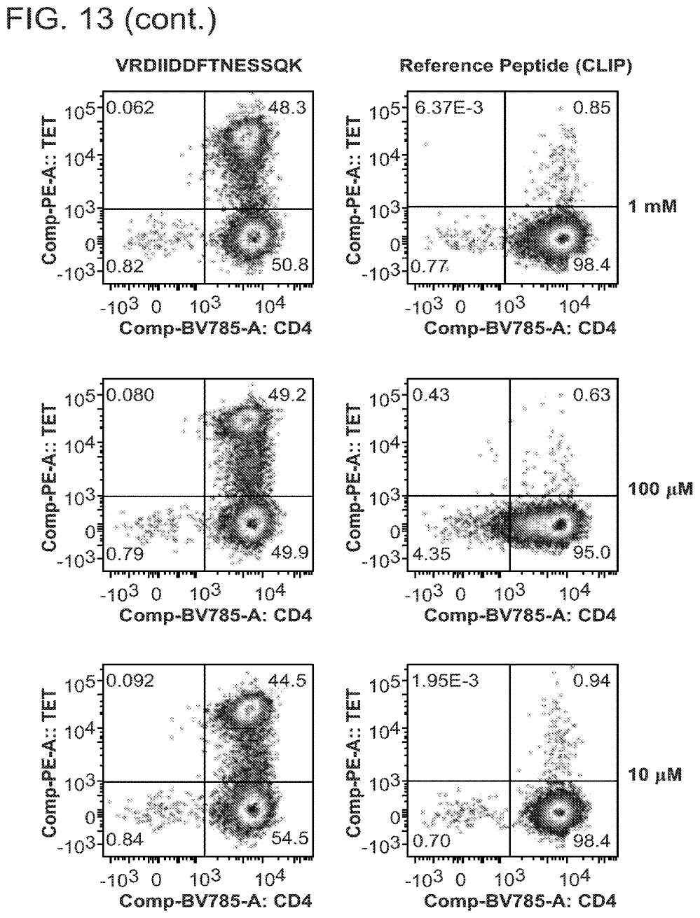

25 μL of HLA-DRB1*04:01 monomer with exiting peptide PK(DNP)PVSLMRMPTPLLM was mixed with 2 μL of emulsifier and 3 μL of 10 mM, 1 mM or 0.1 mM peptides dissolved in 10% DMSO. These peptides were respectively PKYVKQNTLAT (Flu Peptide), TKIYSYFPSVISKV, VRDIIDDFTNESSQK (2 Tetanus Toxin Peptides) and PVSKMRMATPLLMQA (CLIP, negative control). Each peptide exchange monomer (30 μL) was mixed with 10 μL streptavidin-PE. The tetramers were then mixed with 10 μL Neutralizer. PBMCs from a donor expressing HLA-DRB1*04:01 were cultured at 3×105 cells per well and stimulated with peptides. Before staining, cells with incubated with a 10% human AB serum block. Cells were treated with 50 nM Dasatinib and stained with tetramers for 2 hours at 37 C followed by a 20 min staining with anti CD3, CD4 & CD8 antibodies (anti CD3 FITC, anti CD8-BV510, anti CD4 BV785). Note that the percentages of tetramer positive cells are not significantly affected by concentrations of high affinity peptides used in the exchanged reactions. See FIG. 13.

### Example 12: Staining of CD4 Lymphocytes with Peptide Exchanged HLA-DRB1*15:01 PE, APC and BV421 Conjugated Tetramers

25 μL of HLA-DRB1*15:01 monomer with exiting peptide PK(DNP)PVSKYRMATPLLM was mixed with 2 μL of emulsifier and 3 μL of 10 mM peptides dissolved in 10% DMSO. These peptides were respectively PKYVKQNTLAT (Flu Peptide), TKIYSYFPSVISKV, VSTIVPYIGPALNI (2 Tetanus Toxin Peptides) and PVSKMRMATPLLMQA (CLIP, negative control). Each peptide exchange monomer (30 μL) was mixed with 10 μL streptavidin-PE, streptavidin-APC or streptavidin-BV421. The tetramers were then mixed with 10 μL Neutralizer.

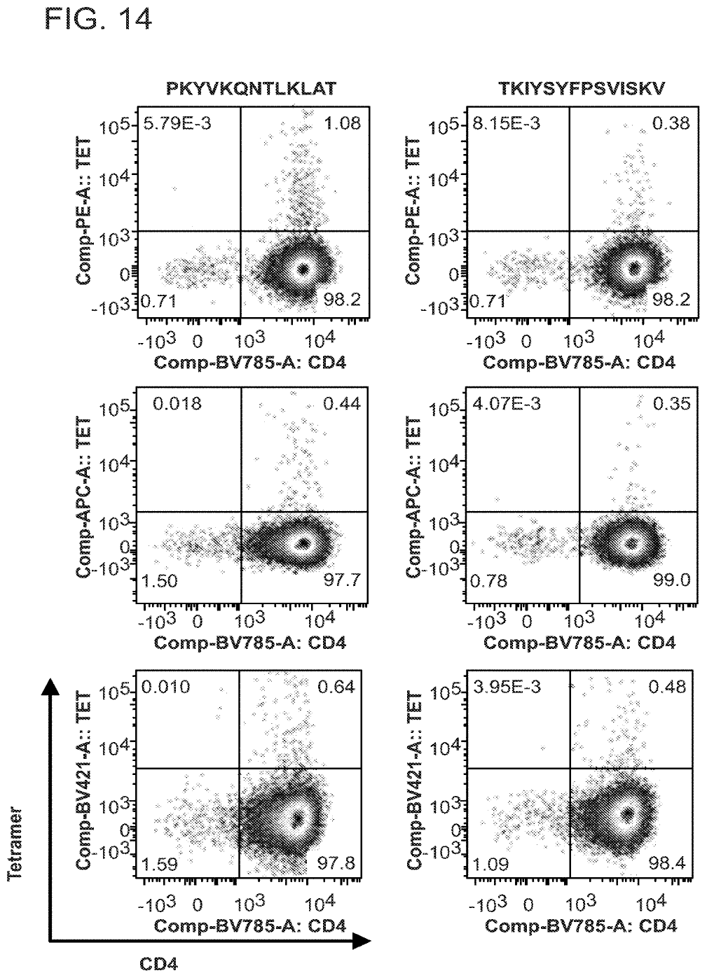

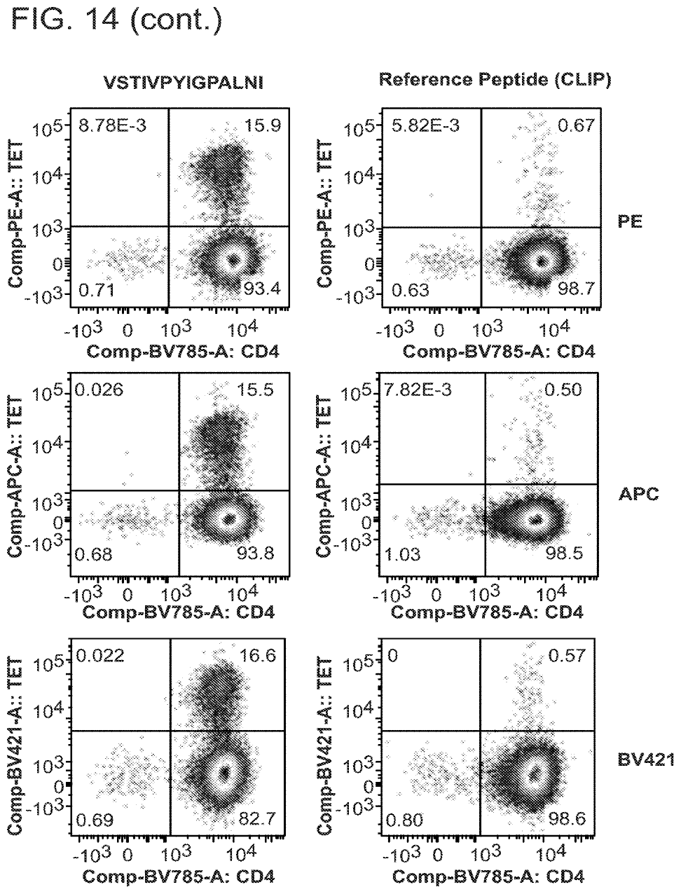

PBMCs from a donor expressing HLA-DRB1*15:01 were cultured at 3×105 cells per well and stimulated with peptides. Before staining, cells with incubated with a 10% human AB serum block. Cells were treated with 50 nM Dasatinib and stained with tetramers for 2 hours at 37 C followed by a 20 min staining with anti CD3, CD4 & CD8 antibodies (anti CD3 FITC, anti CD8-BV510, anti CD4 BV785). Note that the percentages of tetramer positive cells are not significantly affected by the type of fluorochrome conjugated to streptavidin. See FIG. 14.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A protein complex comprising an MHC class II molecule bound to a first peptide, wherein the first peptide is labeled with a first label.

2. The protein complex of claim 1, wherein the first peptide is PKPVSKMRMATPLLM, PKPVSLMRMATPLLM, KPVSKMRMARPLLMQ, PKPVSKYRMATPLLM, or a functional homologue thereof having at least 75% identity with PKPVSKMRMATPLLM, PKPVSLMRMATPLLM, KPVSKMRMARPLLMQ or PKPVSKYRMATPLLM.

3. The protein complex of claim 1 or 2, wherein the first peptide is PKPVSKMRMATPLLM, PKPVSLMRMATPLLM, KPVSKMRMARPLLMQ, PKPVSKYRMATPLLM, or a functional homologue thereof having at least 80% identity with PKPVSKMRMATPLLM, PKPVSLMRMATPLLM, KPVSKMRMARPLLMQ or PKPVSKYRMATPLLM.

4. The protein complex of any preceding claim, wherein the first peptide is PKPVSKMRMATPLLM, PKPVSLMRMATPLLM, KPVSKMRMARPLLMQ, PKPVSKYRMATPLLM, or a functional homologue thereof having at least 90% identity with PKPVSKMRMATPLLM, PKPVSLMRMATPLLM or PKPVSKYRMATPLLM.

5. The protein complex of any preceding claim, wherein the first peptide is PKPVSKMRMATPLLM, PKPVSLMRMATPLLM, KPVSKMRMARPLLMQ, or PKPVSKYRMATPLLM.

6. The protein complex of any preceding claim, wherein the first label is biotin, a hapten, or a tag.

7. The protein complex of any preceding claim, wherein the first label is a first tag.

8. The protein complex of any preceding claim, wherein the first tag is DNP.

9. The protein complex of any one of claims 1-8, wherein the MHC class II molecule is a monomer.

10. The protein complex of any one of claims 1-8, the MHC class II molecule is a component of an MHC class II multimer.

11. The protein complex of claim 10, wherein the MHC class II multimer comprises 4 MHC class II molecules.

12. The protein complex of claim 11, wherein the MHC class II multimer comprises 6 MHC class II molecules.

13. The protein complex of claim 12, wherein the MHC class II multimer comprises 12 MHC class II molecules.

14. The protein complex of any one of claims 10-13, wherein at least one MHC class II molecule of the MHC class II multimer is bound to the first peptide.

15. The protein complex of claim 14, wherein all MHC class II molecules of the MHC class II multimer are bound to the first peptide.

16. The protein complex of any preceding claim, wherein the MHC class II molecule is HLA-DPA1, HLA-DPB1, HLA-DQA1, HLA-DQB1, HLA-DRA, and HLA-DRB1, or I-A, I-E in mice.

17. The protein complex of claim 16, wherein the MHC class II molecule is HLA-DRB1*01:01, HLA-DRB1*04:01 or HLA-DRB1*15:01.

18. The protein complex of any preceding claim, wherein the protein complex can be used for quantified or non-quantified peptide exchange.

19. A kit for peptide exchange comprising an MHC class II molecule bound to a first peptide wherein the first peptide is labeled with a first label.

20. The kit of claim 19, wherein the first peptide is PKPVSKMRMATPLLM, PKPVSLMRMATPLLM, KPVSKMRMARPLLMQ, PKPVSKYRMATPLLM, or a functional homologue thereof having at least 75% identity with PKPVSKMRMATPLLM, PKPVSLMRMATPLLM, KPVSKMRMARPLLMQ, or PKPVSKYRMATPLLM.

21. The kit of claim 19 or 20, wherein the first peptide is PKPVSKMRMATPLLM, PKPVSLMRMATPLLM, KPVSKMRMARPLLMQ, PKPVSKYRMATPLLM, or a functional homologue thereof having at least 80% identity with PKPVSKMRMATPLLM, PKPVSLMRMATPLLM, KPVSKMRMARPLLMQ, or PKPVSKYRMATPLLM.

22. The kit of any one of claims 19-21, wherein the first peptide is PKPVSKMRMATPLLM, PKPVSLMRMATPLLM, KPVSKMRMARPLLMQ, PKPVSKYRMATPLLM, or a functional homologue thereof having at least 90% identity with PKPVSKMRMATPLLM, PKPVSLMRMATPLLM, KPVSKMRMARPLLMQ, or PKPVSKYRMATPLLM.

23. The kit of any one of claims 19-22, wherein the first peptide is PKPVSKMRMATPLLM, PKPVSLMRMATPLLM, KPVSKMRMARPLLMQ, or PKPVSKYRMATPLLM.

24. The kit of any one of claims 19-23, wherein the kit is used for quantified peptide exchange.

25. The kit of any one of claims 19-23, wherein the kit is used for non-quantified peptide exchange.

26. The kit of any one of claims 19-25, wherein the kit does not comprise any catalyst or peptide exchange factor.

27. The kit of any one of claims 19-26, wherein the first peptide is labeled with a first label.

28. The kit of any one of claims 19-27, wherein the first label is biotin, a hapten, or a tag.

29. The kit of any one of claims 19-28, wherein the first label is a first tag.

30. The kit of any one of claims 19-29, wherein the first tag is DNP.

31. The kit of any one of claims 19-30, further comprising an anti-first tag antibody.

32. The kit of any one of claims 19-31, further comprising a capture system comprising an anti-MHC class II protein antibody or a streptavidin.

33. The kit of any one of claims 19-32, wherein the capture system comprises magnetic capture beads.

34. The kit of any one of claims 19-33, further comprising a reference peptide.

35. The kit of any one of claims 19-34, wherein the MHC class II molecule is a monomer.

36. The kit of any one of claims 19-34, wherein the MHC class II molecule is a component of an MHC class II multimer.

37. The kit of claim 36, wherein the multimer comprises 4 MHC class II molecules.

38. The kit of claim 37, wherein the multimer comprises 6 MHC class II molecules.

39. The kit of claim 38, wherein the multimer comprises 12 MHC class II molecules.

40. The kit of any one of claims 19-39, wherein the MHC class II molecule is HLA-DPA1, HLA-DPB1, HLA-DQA1, HLA-DQB1, HLA-DRA, and HLA-DRB1, or I-A, I-E in mice.

41. The kit of claim 40, wherein the MHC class II molecule is HLA-DRB1*01:01, HLA-DRB1*04:01 or HLA-DRB1*15:01.

42. A method of performing peptide exchange on an MHC class II molecule, comprising:
providing an MHC class II molecule bound to a first peptide, wherein the first peptide is labeled with a first label;
contacting the MHC class II molecule bound to the first peptide with a second binding partner, wherein the contacting step generates an MHC class II molecule bound to the second binding partner.

43. The method of claim 42, comprising:
providing a first quantity of MHC class II molecules bound to a first peptide, wherein the first peptide is labeled with a first label;
adding to the MHC class II molecules bound to the first peptide a second quantity of a second binding partner, whereby a mixture is formed comprising MHC class II molecules bound to the first peptide, MHC class II molecules bound to the second binding partner, unbound first peptide, and unbound second binding partner.

44. The method of claim 42 or 43, wherein the first peptide is PKPVSKMRMATPLLM, PKPVSLMRMATPLLM, KPVSKMRMARPLLMQ, PKPVSKYRMATPLLM, or a functional homologue thereof having at least 75% identity with PKPVSKMRMATPLLM, PKPVSLMRMATPLLM, KPVSKMRMARPLLMQ, or PKPVSKYRMATPLLM.

45. The method of any one of claims 42-44, wherein the first peptide is PKPVSKMRMATPLLM, PKPVSLMRMATPLLM, KPVSKMRMARPLLMQ, PKPVSKYRMATPLLM, or a functional homologue thereof having at least 80% identity with PKPVSKMRMATPLLM, PKPVSLMRMATPLLM, KPVSKMRMARPLLMQ, or PKPVSKYRMATPLLM.

46. The method of any one of claims 42-45, wherein the first peptide is PKPVSKMRMATPLLM, PKPVSLMRMATPLLM, PKPVSKYRMATPLLM, KPVSKMRMARPLLMQ, or a functional homologue thereof having at least 90% identity with PKPVSKMRMATPLLM, PKPVSLMRMATPLLM, KPVSKMRMARPLLMQ, or PKPVSKYRMATPLLM.

47. The method of any one of claims 42-46, wherein the first peptide is PKPVSKMRMATPLLM, PKPVSLMRMATPLLM, KPVSKMRMARPLLMQ, or PKPVSKYRMATPLLM.

48. The method of any one of claims 42-47, wherein the method is used for quantified peptide exchange.

49. The method of any one of claims 42-48, wherein the method is used for non-quantified peptide exchange.

50. The method of any one of claims 42-49, wherein the method does not comprise adding any catalyst or peptide exchange factor.

51. The method of any one of claims 42-50, wherein the first peptide is labeled with a first label.

52. The method of any one of claims 42-51, wherein the first label is biotin, a hapten, or a tag.

53. The method of any one of claims 42-52, wherein the first label is a first tag.

54. The method of any one of claims 42-53, wherein the first tag is DNP.

55. The method of any one of claims 42-54, wherein the second binding partner is a second peptide.

56. The method of any one of claims 42-55, wherein the second binding partner or second peptide is not labeled.

57. The method of any one of claims 42-56, wherein the second binding partner or second peptide does not display a tag.

58. The method of any one of claims 42-55, wherein the second binding partner or second peptide displays a second label, and further wherein the first label is different from the second label.

59. The method of claim 58, wherein the second label is selected from biotin, a hapten, or a tag.

60. The method of claim 59, wherein the second label is a second tag, and further wherein the first tag is different from the second tag.

61. The method of any one of claims 42-60, further comprising determining:
(1) at least one of: the amount of MHC class II molecules bound to the first peptide, the amount of MHC class II molecules bound to the second binding partner or second peptide, the amount of unbound first peptide, or the amount of unbound second binding partner or second peptide;
(2) the ratio of the amount of the MHC class II molecules bound to the first peptide to the amount of the MHC class II molecules bound to the second binding partner or second peptide; and/or
(3) the amount of unbound first peptide and comparing the amount of unbound first peptide to the first quantity of MHC class II molecules bound to the first peptide.

62. The method of any one of claims 42-61, wherein the determining step comprises or is accomplished by performing a sandwich immunoassay comprising:
providing a support conjugated to a first antibody or a molecule that binds to an MHC class II molecule;
binding the first antibody or a molecule that binds to an MHC class II molecule to the MHC class II molecules bound to the first peptide and the MHC class II molecules bound to the second binding partner or second peptide;
providing a second antibody capable of binding the first peptide;
binding the second antibody to the MHC class II molecules bound to the first peptide, whereby the second antibody is bound to the support; and
determining the amount of the second antibody that is bound to the support.

63. The method of claim 62, wherein the support comprises magnetic beads, nonmagnetic beads, or microplate wells.

64. The method of claim 62 or 63, wherein performing the sandwich immunoassay comprises pelleting beads using a magnet, by centrifugation, or by suction.

65. The method of any one of claims 62-64, wherein the first antibody is an anti-MHC class II or anti-streptavidin antibody.

66. The method of any one of claims 62-65, wherein the first peptide is labeled with DNP and the second antibody is an anti-DNP antibody.

67. The method of any one of claims 62-66, wherein the second antibody is labeled with a fluorophore, an enzyme, a metal, a barcode, or a radiolabel.

68. The method of any one of claims 62-67, wherein the second antibody is labeled with a fluorophore.

69. The method of any one of claims 42-68, further comprising conducting a control peptide exchange.

70. The method of any one of claims 42-69, wherein the mixture further comprises additional peptides, lipids, or polynucleotides.

71. The method of any one of claims 42-70, wherein the MHC class II molecule is a monomer.

72. The method of any one of claims 42-70, wherein the MHC class II molecule is a component of an MHC class II multimer.

73. The method of claim 72, wherein the multimer comprises 4 MHC class II molecules.

74. The method of claim 73, wherein the multimer comprises 6 MHC class II molecules.

75. The method of claim 74, wherein the multimer comprises 12 MHC class II molecules.

76. The method of any one of claims 42-75, wherein the MHC class II molecule is HLA-DPA1, HLA-DPB1, HLA-DQA1, HLA-DQB1, HLA-DRA, and HLA-DRB1, or I-A, I-E in mice.

77. The method of claim 76, wherein the MHC class II molecule is HLA-DRB1*01:01, HLA-DRB1*04:01 or HLA-DRB1*15:01.


SEQUENCE LISTING (45 sequences)

SEQ ID NO: 1 - protein, 15 aa, Artificial Sequence
  Description of Artificial Sequence Synthetic peptide
PKPVSKMRMATPLLM

SEQ ID NO: 2 - protein, 15 aa, Artificial Sequence
  Description of Artificial Sequence Synthetic peptide
PKPVSLMRMATPLLM

SEQ ID NO: 3 - protein, 15 aa, Artificial Sequence
  Description of Artificial Sequence Synthetic peptide
KPVSKMRMARPLLMQ

SEQ ID NO: 4 - protein, 15 aa, Artificial Sequence
  Description of Artificial Sequence Synthetic peptide
PKPVSKYRMATPLLM

SEQ ID NO: 5 - protein, 15 aa, Artificial Sequence
  Description of Artificial Sequence Synthetic peptide
  MOD_RES (2)..(2): Lys(DNP)
PKPVSKMRMATPLLM

SEQ ID NO: 6 - protein, 15 aa, Artificial Sequence
  Description of Artificial Sequence Synthetic peptide
  MOD_RES (2)..(2): Lys(DNP)
PKPVSLMRMPTPLLM

SEQ ID NO: 7 - protein, 15 aa, Artificial Sequence
  Description of Artificial Sequence Synthetic peptide
  MOD_RES (1)..(1): Lys(DNP)
KPVSKMRMARPLLMQ

SEQ ID NO: 8 - protein, 15 aa, Artificial Sequence
  Description of Artificial Sequence Synthetic peptide
  MOD_RES (2)..(2): Lys(DNP)
PKPVSKYRMATPLLM

SEQ ID NO: 9 - protein, 13 aa, Clostridium tetani
TSLYNLRRGTALA

SEQ ID NO: 10 - protein, 14 aa, Clostridium tetani
VSTIVPYIGPALNI

SEQ ID NO: 11 - protein, 14 aa, Clostridium tetani
QYIKANSKFIGITE

SEQ ID NO: 12 - protein, 14 aa, Clostridium tetani
TKIYSYFPSVISKV

SEQ ID NO: 13 - protein, 13 aa, Influenza A virus
PKYVKQNTLKLAT

SEQ ID NO: 14 - protein, 15 aa, Artificial Sequence
  Description of Artificial Sequence Synthetic peptide
PVSKMRMATPLLMQA

SEQ ID NO: 15 - protein, 14 aa, Clostridium tetani
TKIYSYFPSVISKV

SEQ ID NO: 16 - protein, 11 aa, Unknown
  Description of Unknown Influenza virus sequence
PKYVKQNTLAT

SEQ ID NO: 17 - protein, 15 aa, Clostridium tetani
VRDIIDDFTNESSQK

SEQ ID NO: 18 - protein, 15 aa, Artificial Sequence
  Description of Artificial Sequence Synthetic peptide
AGFFLLTRILTIPQS

SEQ ID NO: 19 - protein, 13 aa, Artificial Sequence
  Description of Artificial Sequence Synthetic peptide
AKFVAAWTLKAAA

SEQ ID NO: 20 - protein, 16 aa, Artificial Sequence
  Description of Artificial Sequence Synthetic peptide
DLTFIAEKNSFSSEEP

SEQ ID NO: 21 - protein, 16 aa, Artificial Sequence
  Description of Artificial Sequence Synthetic peptide
DRFYKTLRAEQASQEV

SEQ ID NO: 22 - protein, 15 aa, Artificial Sequence
  Description of Artificial Sequence Synthetic peptide
ENPVVHFFKNIVTPR

SEQ ID NO: 23 - protein, 15 aa, Artificial Sequence
  Description of Artificial Sequence Synthetic peptide
FHTYTIDWTKDAVTW

SEQ ID NO: 24 - protein, 21 aa, Artificial Sequence
  Description of Artificial Sequence Synthetic peptide
FNNFTVSFWLRVPKVSASHLE

SEQ ID NO: 25 - protein, 14 aa, Artificial Sequence
  Description of Artificial Sequence Synthetic peptide
GIAGFKGEQGPKGE

SEQ ID NO: 26 - protein, 15 aa, Artificial Sequence
  Description of Artificial Sequence Synthetic peptide
GIAGFKGEQGPKGEP

SEQ ID NO: 27 - protein, 15 aa, Artificial Sequence
  Description of Artificial Sequence Synthetic peptide
GINAVAYYRGLDVSV

SEQ ID NO: 28 - protein, 15 aa, Artificial Sequence
  Description of Artificial Sequence Synthetic peptide
GYKVLVLNPSVAATL

SEQ ID NO: 29 - protein, 15 aa, Artificial Sequence
  Description of Artificial Sequence Synthetic peptide
KKQFEELTLGEFLKL

SEQ ID NO: 30 - protein, 15 aa, Artificial Sequence
  Description of Artificial Sequence Synthetic peptide
KRWIILGLNKIVRMY

SEQ ID NO: 31 - protein, 14 aa, Artificial Sequence
  Description of Artificial Sequence Synthetic peptide
LPLKMLNIPSINVH

SEQ ID NO: 32 - protein, 19 aa, Artificial Sequence
  Description of Artificial Sequence Synthetic peptide
NPVVHFFKNIVTPRTPPPQ

SEQ ID NO: 33 - protein, 15 aa, Artificial Sequence
  Description of Artificial Sequence Synthetic peptide
PIPIHYCAPAGFAIL

SEQ ID NO: 34 - protein, 15 aa, Artificial Sequence
  Description of Artificial Sequence Synthetic peptide
RELERFAVNPGLLET

SEQ ID NO: 35 - protein, 21 aa, Artificial Sequence
  Description of Artificial Sequence Synthetic peptide
SGIQYLAGLSTLPGNPAIASL

SEQ ID NO: 36 - protein, 15 aa, Artificial Sequence
  Description of Artificial Sequence Synthetic peptide
SLQPLALEGSLQSRG

SEQ ID NO: 37 - protein, 15 aa, Artificial Sequence
  Description of Artificial Sequence Synthetic peptide
TENFNMWKNNMVEQM

SEQ ID NO: 38 - protein, 14 aa, Artificial Sequence
  Description of Artificial Sequence Synthetic peptide
VNFFRMVISNPAAT

SEQ ID NO: 39 - protein, 15 aa, Artificial Sequence
  Description of Artificial Sequence Synthetic peptide
YALFYKLDVVPIDND

SEQ ID NO: 40 - protein, 20 aa, Artificial Sequence
  Description of Artificial Sequence Synthetic peptide
WITQCFLPVFLAQPPSGQRR

SEQ ID NO: 41 - protein, 18 aa, Artificial Sequence
  Description of Artificial Sequence Synthetic peptide
ISNQLTLDSNTKYFHKLN

SEQ ID NO: 42 - protein, 4 aa, Artificial Sequence
  Description of Artificial Sequence Synthetic peptide
TSLY

SEQ ID NO: 43 - protein, 4 aa, Artificial Sequence
  Description of Artificial Sequence Synthetic peptide
QYIK

SEQ ID NO: 44 - protein, 4 aa, Artificial Sequence
  Description of Artificial Sequence Synthetic peptide
TKIY

SEQ ID NO: 45 - protein, 4 aa, Artificial Sequence
  Description of Artificial Sequence Synthetic peptide
VSTI